(19) United States
(12) Patent Application Publication
(10) Pub. No.: US 2026/0338889 A1
(43) Pub. Date: Oct. 29, 2026

(54) DATA TRANSMISSION SYSTEMS AND METHODS

(71) Applicant: SHANGHAI UNITED IMAGING HEALTHCARE CO., LTD., Shanghai (CN)

(72) Inventors: Dongming WANG, Shanghai (CN); Fangyan HOU, Shanghai (CN); Yingdong WU, Shanghai (CN)

(73) Assignee: SHANGHAI UNITED IMAGING HEALTHCARE CO., LTD., Shanghai (CN)

(21) Appl. No.: 19/698,952

(22) Filed: Jun. 5, 2026

Related U.S. Application Data
(63) Continuation of application No. PCT/CN2024/143364, filed on Dec. 27, 2024.

(30) Foreign Application Priority Data
Dec. 29, 2023 (CN) 202311872280.0

(51) Int. Cl. G01R 33/36; G01R 33/54

(52) U.S. Cl. CPC G01R 33/3692; G01R 33/54

(57) ABSTRACT

A data transmission system for MR imaging is provided. The system comprises: a signal converting device configured to receive analog MR signals collected by a receiver coil in an MR scan, transform the analog MR signals into digital MR signals, generate transmission data based on the digital MR signals, and transmit the transmission data to a signal processing device via a first communication link between the signal converting device and the signal processing device; and the signal processing device configured to receive the transmission data from the signal converting device via the first communication link and resolve the digital MR signals from the transmission data, wherein the signal converting device and the signal processing device are disposed at different circuit boards.

### CROSS-REFERENCE TO RELATED APPLICATIONS

This application is a continuation of international patent application No. PCT/CN2024/143364 filed on Dec. 27, 2024, which claims priority to Chinese Patent Application No. 202311872280.0 filed on Dec. 29, 2023, the entire contents of each of which are incorporated herein by reference.

### TECHNICAL FIELD

The present disclosure generally relates to magnetic resonance (MR) imaging filed, and in particular, to data transmission systems and methods for MR imaging.

### BACKGROUND

MR imaging technology has been widely used for generating medical images of interior of a patient's body for, e.g., clinical examinations, medical diagnosis, and/or treatment purposes. Since a large amount of data is generated during an MR scan, and an efficient data transmission system is needed for MR imaging.

### SUMMARY

A data transmission system for MR imaging is provided. The system comprises: a signal converting device configured to receive analog MR signals collected by a receiver coil in an MR scan, transform the analog MR signals into digital MR signals, generate transmission data based on the digital MR signals, and transmit the transmission data to a signal processing device via a first communication link between the signal converting device and the signal processing device; and the signal processing device configured to receive the transmission data from the signal converting device via the first communication link and resolve the digital MR signals from the transmission data, wherein the signal converting device and the signal processing device are disposed at different circuit boards.

In some embodiments, to establish the first communication link, the signal processing device is configured to transmit a clock synchronization instruction to the signal converting device via a second communication link between the signal converting device and the signal processing device. In response to the clock synchronization instruction, the signal converting device is configured to perform clock synchronization on one or more analog-to-digital converters (ADCs) of the signal converting device and transmit a connection request to the signal processing device after the clock synchronization is achieved. The signal processing device is configured to transmit a connection instruction to the signal converting device in response to the connection request, or the signal converting device is configured to generate the connection instruction. In response to the connection instruction, the signal converting device is configured to generate link data and transmit the link data to the signal processing device, the signal processing device is configured to confirm whether the first communication link is established based on the link data.

In some embodiments, the connection instruction is generated by the signal processing device and transmitted to the signal converting device.

In some embodiments, the clock synchronization instruction is transmitted in the form of a clock signal. The signal processing device includes a clock source configured to generate the clock signal. The signal converting device includes a clock synchronizer. The second communication link is established between the clock source and the clock synchronizer. The clock synchronizer is configured to receive the clock signal via the second communication link and perform the clock synchronization on the one or more ADCs based on the clock signal.

In some embodiments, to perform the clock synchronization on the one or more ADCs, the clock synchronizer is further configured to: for each of the one or more ADCs, generate a second clock signal corresponding to the ADC based on the clock signal; and transmit the second clock signal corresponding to the ADC to the ADC for performing the clock synchronization.

In some embodiments, the signal converting device includes: a configurator connected to the signal processing device via a third communication link between the signal converting device and the signal processing device and configured to receive the connection instruction from the signal processing device via the third communication link; the one or more ADCs connected to the configurator. To generate the link data, the configurator is configured to generate one or more synchronization signals in response to the connection instruction, and transmit the one or more synchronization signals to the one or more ADCs, the one or more ADCs are configured to generate the link data in response to the one or more synchronization signals.

In some embodiments, the signal converting device includes a data transmitter connected to the one or more ADCs. The signal processing device includes a data receiver, the first communication link is achieved by a wired or wireless connection between the data transmitter and the data receiver. To generate the link data, the one or more ADCs are configured to obtain initial data, adjust the format and/or the phase of the initial data, and transform the adjusted initial data into the link data based on a digital protocol of the data receiver.

In some embodiments, the initial data are target digital MR signals converted from a portion of the analog MR signals.

In some embodiments, the signal converting device further comprises one or more analog signal processors each of which is connected to a receiving coil, the configurator, and one of the one or more ADCs. The configurator is configured to receive a signal preprocessing instruction including one or more operating parameter values of the one or more analog signal processors from the signal processing device, and transmit the one or more operating parameter values to the one or more analog signal processors. The one or more analog signal processors are configured to generate preprocessed analog MR signals by preprocessing the portion of the analog MR signals based on the one or more operating parameter values, and transmit the preprocessed analog MR signals to the one or more ADCs. To convert the portion of the analog MR signals into the target digital MR signals, the one or more ADCs are configured to receive the preprocessed analog MR signals from the one or more analog signal processors and convert the preprocessed analog MR signals into the target digital MR signals.

In some embodiments, the clock synchronization instruction is transmitted in the form of high-speed data. The signal processing device includes a clock source and a data processor connected to the clock source, the clock source being configured to generate a clock signal, and the data processor being configured to generate the high-speed data based on the clock signal. The signal converting device includes a configurator, the second communication link is established between the data processor and the configurator, and the configurator is configured to receive the high-speed data via the second communication link, restore the clock signal from the high-speed data, and perform the clock synchronization on the one or more ADCs based on the clock signal.

In some embodiments, the signal converting device further comprises one or more analog signal processors each of which is connected to a receiving coil, the configurator, and one of the one or more ADCs. The high-speed data further includes one or more operating parameter values of the one or more analog signal processors. The configurator is further configured to restore the one or more operating parameter values from the high-speed data, and transmit the one or more operating parameter values to the one or more analog signal processors.

In some embodiments, the signal converting device is further configured to start timing in response to the clock synchronization instruction. The connection instruction is generated by the signal converting device after a first preset period. The signal processing device is further configured to start timing in response to the connection request, and generate a second connection instruction corresponding to the connection instruction after a second preset period.

In some embodiments, the clock synchronization instruction is transmitted in the form of a clock signal. The signal processing device includes a clock source configured to generate the clock signal. The signal converting device includes a clock synchronizer and a configurator connected to the clock synchronizer. The second communication link is established between the clock source and the clock synchronizer. The clock synchronizer is configured to receive the clock signal from the clock source via the second communication link, perform the clock synchronization on the one or more ADCs based on the clock signal, and transmit a clock-locking signal to the configurator. The configurator is configured to start timing in response to the clock-locking signal and generate the connection instruction after the first preset period.

In some embodiments, to generate the link data, the configurator is configured to transmit the connection instruction to each of the one or more ADCs, and the one or more ADCs are configured to generate the link data in response to the connection instruction.

In some embodiments, the clock synchronizer is a phase-locked loop, the one or more ADCs include one multichannel ADC, the signal converting device further comprises one or more analog signal processors each of which includes a frequency mixer and is connected to a receiving coil, the phase-locked loop, and the multichannel ADC. The phase-locked loop is configured to adjust a mixing frequency of the frequency mixer of each analog signal processor such that the frequency of preprocessed analog MR signals generated by each analog signal processor is within a preset frequency range corresponding to the multichannel ADC.

In some embodiments, the clock synchronization instruction is transmitted in the form of high-speed data. The signal processing device includes a clock source and a data processor connected to the clock source, the clock source being configured to generate a clock signal, and the data processor being configured to generate the high-speed data based on the clock signal. The signal converting device includes a clock synchronizer and a configurator connected to the clock synchronizer. The second communication link is established between the configurator and the data processor. The configurator is configured to receive the high-speed data via the second communication link, restore the clock signal from the high-speed data, and transmit the clock signal to the clock synchronizer. The clock synchronizer is configured to perform the clock synchronization on the one or more ADCs based on the clock signal and transmit a clock-locking signal to the configurator. The configurator is further configured to start timing in response to the clock-locking signal and generate the connection instruction after the first preset period.

In some embodiments, the signal converting device further comprises one or more analog signal processors each of which is connected to a receiving coil, the configurator, and one of the one or more ADCs. The high-speed data further includes one or more operating parameter values of the one or more analog signal processors. The configurator is further configured to restore the one or more operating parameter values from the high-speed data, and transmit the one or more operating parameter values to the one or more analog signal processors.

In some embodiments, before the first communication link is established, the signal processing device is configured to transmit a clock synchronization instruction to the signal converting device via a second communication link between signal converting device and the signal processing device. In response to the clock synchronization instruction, the signal converting device is configured to start timing, perform clock synchronization on one or more ADCs, and transmit a connection request to the signal processing device after the clock synchronization is achieved. After a first preset period, the signal converting device is further configured to generate a connection instruction, generate link data in response to the connection instruction, and transmit the link data to the signal processing device. The signal processing device is configured to start timing in response to the connection request, generate a second connection instruction corresponding to the connection instruction after a second preset period, and confirm whether the first communication link is established based on the link data.

In some embodiments, the signal converting device is disposed on or within a couch or a receiver coil of an MR device.

In some embodiments, the signal processing device is disposed on or within a gantry of an MR device.

In some embodiments, the transmission data is transmitted via the first communication link using optical waves or millimeter waves.

A method for establishing a first communication link in MR imaging is provided. The method comprises: by a signal processing device, transmitting a clock synchronization instruction to a signal converting device via a second communication link between the signal converting device and the signal processing device; by the signal converting device, performing clock synchronization on one or more analog-to-digital converters (ADCs) of the signal converting device and transmitting a connection request to the signal processing device after the clock synchronization is achieved; transmitting a connection instruction from the signal processing device to the signal converting device in response to the connection request, or generating the connection instruction by the signal converting device; by the signal converting device, generating link data in response to the connection instruction and transmitting the link data to the signal processing device; by the signal processing device, confirming whether the first communication link is established based on the link data.

In some embodiments, the signal converting device and the signal processing device are disposed on different circuit boards.

In some embodiments, the connection instruction is generated by the signal processing device and transmitted to the signal converting device.

In some embodiments, the connection instruction is generated by the signal converting device by: starting timing in response to the clock synchronization instruction; generating the connection instruction after a first period.

In some embodiments, the method further comprises: by the signal processing device, starting timing in response to the connection request; and generating a second connection instruction corresponding to the connection instruction after a second preset period.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1A is a schematic diagram illustrating an exemplary data transmission system according to some embodiments of the present disclosure;

FIG. 1B is a schematic diagram illustrating another exemplary data transmission system according to some embodiments of the present disclosure;

FIGS. 1C and 1D are schematic diagrams illustrating exemplary data transmission systems, respectively, according to some embodiments of the present disclosure;

FIG. 1E is a schematic diagram illustrating exemplary positions of a signal converting device and a signal processing device on an MR device according to some embodiments of the present disclosure;

FIG. 1F is a schematic diagram illustrating exemplary positions of a signal converting device on a couch of an MR device according to some embodiments of the present disclosure;

FIG. 2 is a flowchart illustrating an exemplary process for establishing a first communication link according to some embodiments of the present disclosure;

FIG. 3 is a schematic diagram illustrating an exemplary data transmission system according to some embodiments of the present disclosure;

FIG. 4 is a schematic diagram illustrating an exemplary data transmission system according to some embodiments of the present disclosure;

FIGS. 5A and 5B are flowcharts illustrating an exemplary process for establishing a first communication link according to some embodiments of the present disclosure;

FIG. 6A is a schematic diagram illustrating an exemplary data transmission system according to some embodiments of the present disclosure;

FIG. 6B is a schematic diagram illustrating an exemplary data transmission system according to some embodiments of the present disclosure;

FIGS. 7A and 7B are flowcharts illustrating an exemplary process for establishing a first communication link according to some embodiments of the present disclosure;

FIG. 8 is a flowchart illustrating an exemplary process for establishing a first communication link according to some embodiments of the present disclosure;

FIG. 9 is a schematic diagram illustrating an exemplary data transmission system according to some embodiments of the present disclosure;

FIG. 10 is a schematic diagram illustrating an exemplary data transmission system according to some embodiments of the present disclosure;

FIGS. 11A and 11B are flowcharts illustrating an exemplary process for establishing a first communication link according to some embodiments of the present disclosure;

FIG. 12 A is a schematic diagram illustrating an exemplary data transmission system according to some embodiments of the present disclosure;

FIG. 12B is a schematic diagram illustrating an exemplary data transmission system according to some embodiments of the present disclosure; and

FIGS. 13A and 13B are flowcharts illustrating an exemplary process for establishing a first communication link according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assembly of different levels in ascending order. However, the terms may be displaced by another expression if they achieve the same purpose.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

It will be understood that, although the terms “first,” “second,” “third,” “fourth,” etc., may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention.

Spatial and functional relationships between elements (for example, between crystal elements) are described using various terms, including “connected,” “engaged,” “interfaced,” and “coupled.” Unless explicitly described as being “direct,” when a relationship between first and second elements is described in the present disclosure, that relationship includes a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.). As used herein, the term “and/or” includes any and all combinations of one or more of the associated listed items.

An anatomical structure shown in an image of a subject (e.g., a patient) may correspond to an actual anatomical structure existing in or on the subject's body. The term “object” and “subject” in the present disclosure are used interchangeably to refer to a biological object (e.g., a patient, an animal) or a non-biological object (e.g., a phantom). In some embodiments, the object may include a specific part, organ, and/or tissue of the object. For example, the object may include the head, the bladder, the brain, the neck, the torso, a shoulder, an arm, the thorax, the heart, the stomach, a blood vessel, soft tissue, a knee, a foot, or the like, or any combination thereof, of a patient.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

Usually, a data transmission system for MR imaging includes a signal converting device and a signal processing device, and transmits data via a communication link between the signal converting device and the signal processing device.

Conventionally, the signal converting device and the signal processing device need to be tightly integrated, often on the same circuit board, to achieve high data transmission rate. However, this limits the flexibility of arrangement of the signal converting device and the signal processing device, resulting in some problems such as increasing a volume, power consumption and a cost of an MR device, and making heat dissipation processing more difficult.

An aspect of the present disclosure provides a data transmission system for MR imaging. The data transmission system may include a signal converting device and a signal processing device. The signal converting device may be configured to receive analog MR signals collected by a receiver coil in an MR scan, transform the analog MR signals into digital MR signals, generate transmission data based on the digital MR signals, and transmit the transmission data to the signal processing device via a first communication link between the signal converting device and the signal processing device. The signal processing device may be configured to receive the transmission data from the signal converting device via the first communication link and resolve the digital MR signals from the transmission data. The signal converting device and the signal processing device may be disposed at different circuit boards.

Compared with the conventional data transmission systems, the signal converting device and the signal processing device in the data transmission systems and methods of the present disclosure do not need to be tightly integrated, which may improve the flexibility of arrangement of the signal converting device and the signal processing device while achieve high data transmission rate. In this way, the volume, the power consumption, and the cost of the MR device may be reduced, and the heat dissipation processing is easier, thereby having a high practicability.

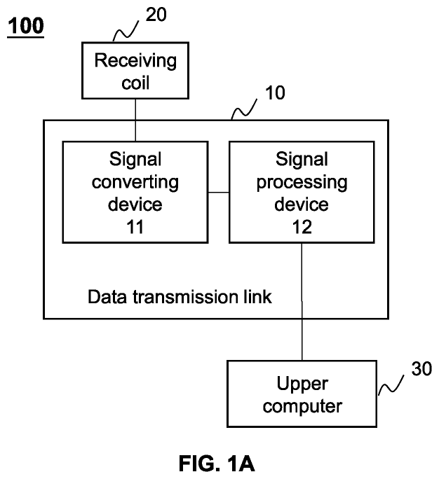

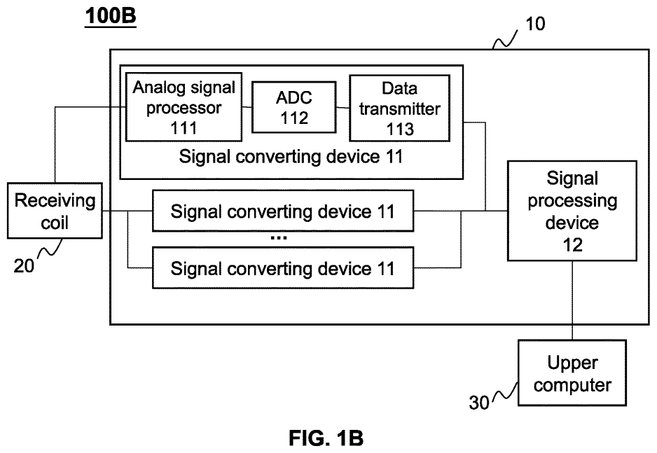

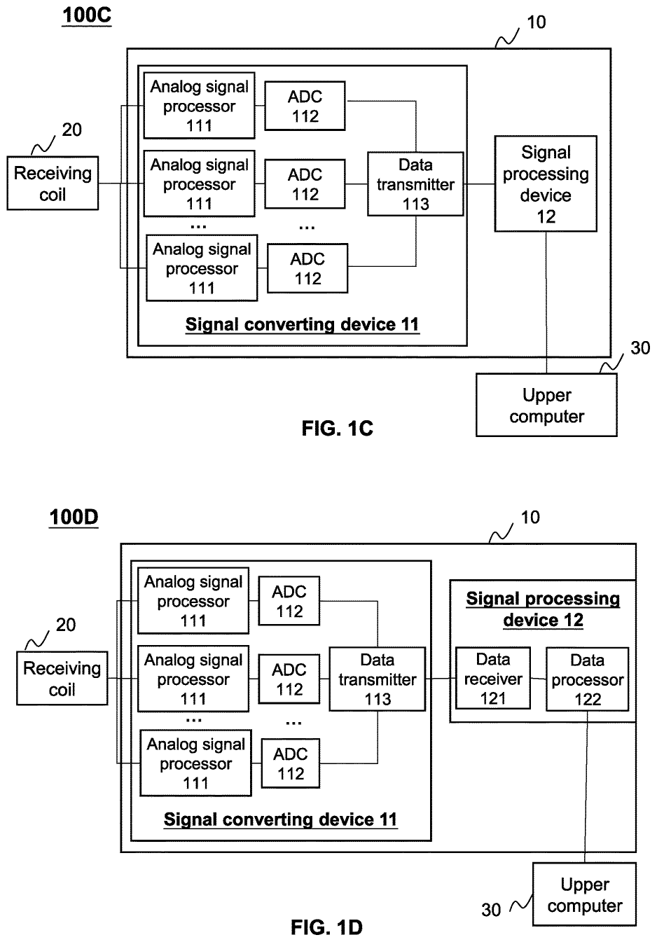

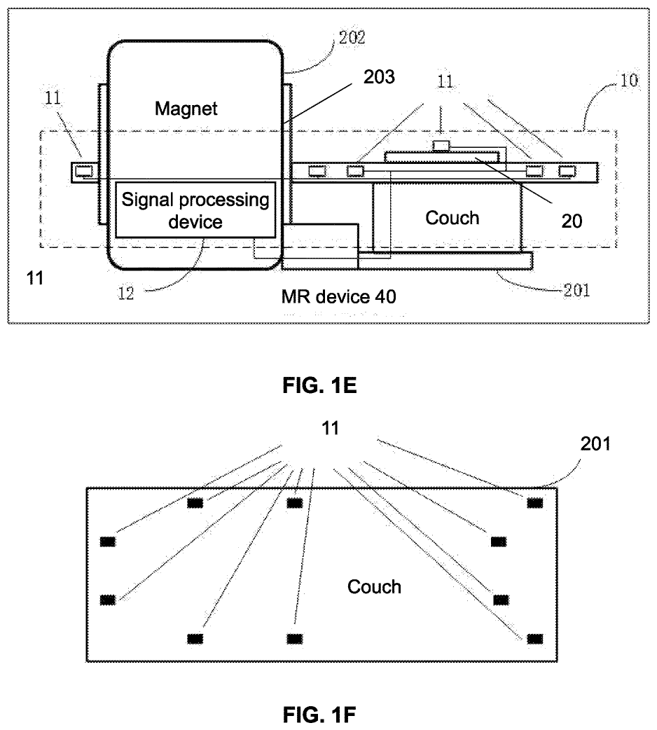

FIG. 1A is a schematic diagram illustrating an exemplary data transmission system 100 according to some embodiments of the present disclosure.

In some embodiments, as illustrated in FIG. 1A, the data transmission system 100 may include a data transmission link 10, a receiving coil 20, and an upper computer 30. The components of the data transmission system 100 may be communicatively connected in various manners.

The receiving coil 20 may be configured to collect MR signals (also referred to as analog MR signals) from a subject in an MR scan of the subject, and transmit the analog MR signals to the upper computer 30 via the data transmission link 10. The receiving coil 20 may be a radiofrequency (RF) receiver coil.

The data transmission link 10 may be configured to transmit the MR signals from the receiving coil 20 to the upper computer 30. In some embodiments, the data transmission link 10 may include a signal converting device 11 and a signal processing device 12. The signal converting device 11 may be connected to the receiving coil 20 and the signal processing device 12, respectively. The signal processing device 12 may be connected to the upper computer 30 and the signal processing device 12, respectively.

In some embodiments, the data transmission link 10 may include one or more signal converting devices 11. For example, FIG. 1B is a schematic diagram illustrating another exemplary data transmission system 100B according to some embodiments of the present disclosure. As shown in FIG. 1B, the data transmission link 10 may include a plurality of signal converting devices 11 and a signal processing device 12. Each signal converting device 11 may be connected to the signal processing device 12. In some embodiments, each signal converting device 11 may include one or more analog signal processors 111, one or more analog-to-digital converters (ADCs) 112, and a data transmitter 113. For example, as shown in FIG. 1B, each signal converting device 11 may include one analog signal processor 111, one ADC 112, and one data transmitter 113. The analog signal processor 111 may be connected to the receiving coil 20 and the ADC 112, respectively. The data transmitter 113 may be connected to the ADC 112 and the signal processing device 12.

As another example, FIGS. 1C and 1D are schematic diagrams illustrating exemplary data transmission system 100C and 100D, respectively, according to some embodiments of the present disclosure. As shown in FIGS. 1C and 1D, the data transmission link 10 may one signal converting device 11 and one signal processing device 12. The signal converting device 11 may include analog signal processors 111, ADCs 112, and one data transmitter 113. Each analog signal processor 111 may be connected to the receiving coil 20 and one of the ADCs 112, respectively. The data transmitter 113 may be connected to each ADC 112 and the signal processing device 12.

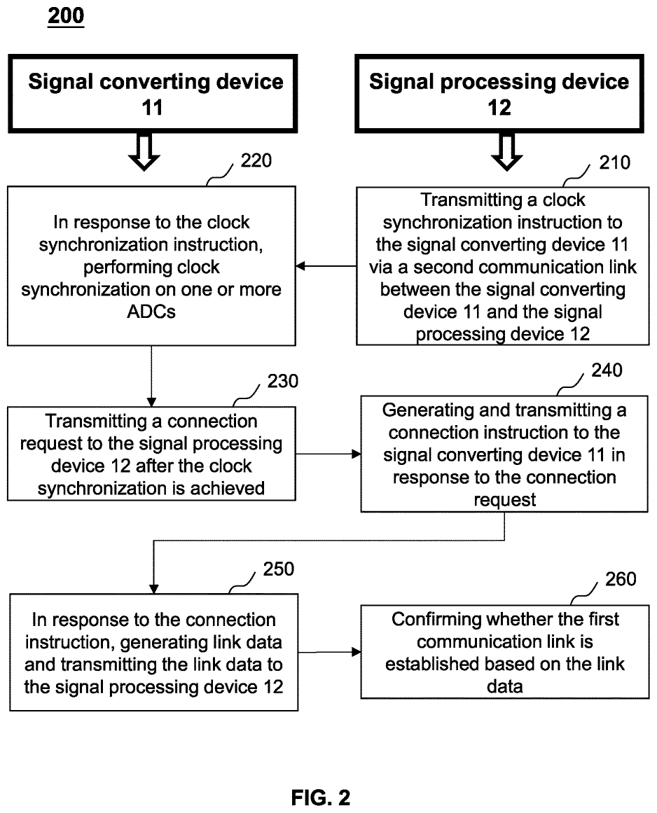

In some embodiments, as shown in FIG. 1D, the signal processing device 12 may include a data receiver 121 and a data processor 122. The data receiver 121 may be connected to the data transmitter 113 and the data processor 122, respectively. The data processor 122 may be further connected to the upper computer 30. More descriptions regarding the analog signal processor 111, the ADC 112, the data transmitter 113, the data receiver 121, and the data processor 122 may be found elsewhere in the present disclosure. See, e.g., FIGS. 2-13B and relevant descriptions thereof.

The signal converting device 11 may be configured to receive the analog MR signals collected by the receiver coil 20 in the MR scan, transform the analog MR signals into digital MR signals, generate transmission data based on the digital MR signals, and transmit the transmission data to the signal processing device 12 via a first communication link between the signal converting device 11 and the signal processing device 12 (as indicated by a line connecting each signal converting device 11 and the signal processing device 12 in FIGS. 1A-1D). The first communication link may be used to transmit the transmission data generated based the analog MR signals collected during the MR scan. In some embodiments, the signal converting device 11 may be configured to perform one or more preprocessing operations such as signal filtering operation, gain operation when processing the analog MR signals, etc.

The signal processing device 12 may be configured to receive the transmission data from the signal converting device 11 via the first communication link and resolve the digital MR signals from the transmission data. In some embodiments, the signal processing device 12 may further transmit the digital MR signals to the upper computer 30. Optionally, the signal processing device 12 may further process the digital MR signals and send the processed digital MR signals to the upper computer 30. Exemplary processing operations may include a filtering, a frequency conversion operation, an up and down conversion operation, an extraction operation, an interpolation operation, a combination operation, an analysis and compression operation, etc. In some embodiments, the signal processing device 12 may process the digital MR signals using an artificial intelligence (AI) algorithm such as a machine learning algorithm (e.g., a neural network algorithm, a deep learning algorithm). In some embodiments, the upper computer 30 may reconstruct an MR image based on the digital MR signals or the processed digital MR signals.

In some embodiments, the signal converting device 11 and the signal processing device 12 may be disposed at different circuit boards.

In some embodiments, the first communication link may be achieved by a wired connection between the signal converting device 11 and the signal processing device 12. For example, the transmission data may be transmitted via the first communication link using RF cables such as coaxial cables, optical fibers, etc. In some embodiments, the first communication link may be achieved by a wireless connection between the signal converting device 11 and the signal processing device 12. For example, the transmission data may be transmitted via the first communication link using optical waves, millimeter waves, lasers, etc. According to some embodiments of the present disclosure, the first communication link may be achieved by the wireless connection between the signal converting device 11 and the signal processing device 12, that is, the data transmission can be performed without the use of cables, which may reduce or avoid signal interference, thereby improving the quality and accuracy of MR images and reducing the cost of the MR device.

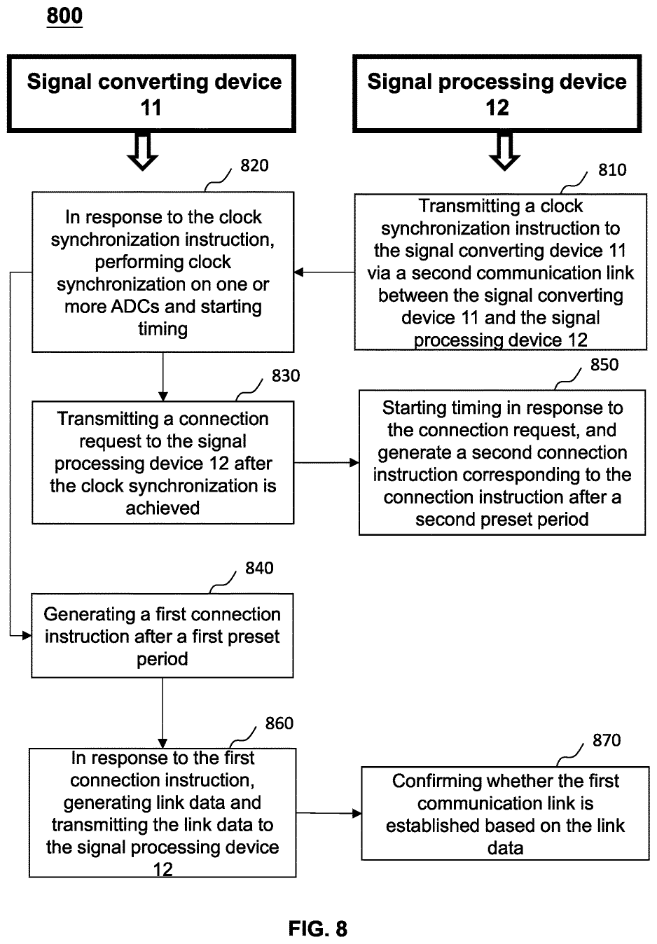

In some embodiments, to establish the first communication link, the signal converting device 11 and the signal processing device 12 may be configured to perform the following operations. The signal processing device 12 may be configured to transmit a clock synchronization instruction to the signal converting device 11 via a second communication link between the signal converting device 11 and the signal processing device 12. In response to the clock synchronization instruction, the signal converting device 11 may be configured to perform clock synchronization on one or more analog-to-digital converters (ADCs) of the signal converting device 11 and transmit a connection request to the signal processing device 12 after the clock synchronization is achieved. The signal converting device 11 may be configured to obtain a connection instruction. In response to the connection instruction, the signal converting device 11 may be configured to generate link data and transmit the link data to the signal processing device 12. The signal processing device 12 may be configured to confirm whether the first communication link is established based on the link data. In some embodiments, in response to the connection request, the signal processing device 12 may be configured to generate the connection instruction and transmit the connection instruction to the signal converting device 11. That is, the first communication link may be established based on the connection instruction generated by the signal processing device 12. More descriptions regarding the establishing of the first communication link based on the connection instruction generated by the signal processing device 12 may be found elsewhere in the present disclosure. See, e.g., FIGS. 2-7B and relevant descriptions thereof. In some embodiments, the signal converting device 11 may be configured to generate the connection instruction. That is, the first communication link may be established based on the connection instruction generated by the signal converting device 11. More descriptions regarding the establishing of the first communication link based on the connection instruction generated by the signal converting device 11 may be found elsewhere in the present disclosure. See, e.g., FIGS. 8-13B and relevant descriptions thereof.

In some embodiments, during the establishment of the first communication link, the signal conversion module 11 needs to be configured. After the first communication link is established, the signal conversion module 11 and the signal processing device 12 remain connected.

In some embodiments, a data transmission system disclosed herein (e.g., data transmission systems 100, 300, 400, 600A, 600B, 900, 1000, 1200A, 1200B) may execute instructions and accordingly be directed to perform one or more processes (e.g., processes 200, 500, 700, 800, 1100, and 1300) described in the present disclosure.

In some embodiments, the signal converting device 11 and the signal processing device 12 may be disposed on an MR device. For example, FIG. 1E is a schematic diagram illustrating exemplary positions of the signal converting device 11 and the signal processing device 12 on an MR device according to some embodiments of the present disclosure. FIG. 1F is a schematic diagram illustrating exemplary positions of the signal converting device 11 on a couch of an MR device according to some embodiments of the present disclosure. As shown in FIGS. 1E and 1F, the MR device 40 may include a couch 201, a magnet 202, a gantry 203, and a receiving coil 20, etc. The gantry 203 may be annular to form a cavity. The couch 201 may be used to support the receiving coil 20. The receive coil 20 may move with the couch 201 in the cavity. The data transmission system 100 may include multiple signal converting devices 11. The signal converting devices 11 may be disposed on or within the couch 201 or the receiver coil 20 of the MR device 40. For example, the signal converting device 11 (e.g., the signal converting device 11 of the data transmission system 400) may be disposed on or within the couch 201 or the receiver coil 20 of the MR device 40. In this case, the MR device 40 may be used to perform a single-radionuclide scan or and a multi-radionuclide scan. As another example, the signal converting device 11 (e.g., the signal converting device 11 of the data transmission system 1000) may be disposed on or within the receiver coil 20 of the MR device 40. In this case, the MR device 40 may be used to perform the single-radionuclide scan. The signal processing device 12 may be disposed on or within the gantry 203 of the MR device 40. In some embodiments, the magnet 202 may be disposed within the gantry 203, and the signal processing device 12 may be disposed on or within the magnet 202. By arranging the signal converting devices 11 on or within the couch 201, or arranging the signal converting devices 11 with low power consumption and a small size on or within the receiving coil 20, the heat dissipation of a circuit board may be improved and the size of the circuit board may be reduced, thereby reducing the requirements for chip integration. By arranging the signal processing device 12 with high power consumption and a large size on or within the magnet 202, air cooling, water cooling, etc. may be used for heat dissipation, which can improve the efficiency of data processing of the signal processing device 12.

It should be noted that the above description of the data transmission system 100 is merely provided for the purposes of illustration, and not intended to limit the scope of the present disclosure. For persons having ordinary skills in the art, multiple variations and modifications may be made under the teachings of the present disclosure. For example, the data transmission system 100 may include one or more additional components and/or one or more components of the data transmission system 100 described above may be omitted. For example, the data transmission system 100 may include a network configured to facilitate exchange of information and/or data. In some embodiments, one or more components (e.g., the signal converting device 11, the signal processing device 12, the receiving coil 20, and the upper computer 30) of the data transmission system 100 may send information and/or data to other component(s) of the data transmission system 100 via the network. As another example, the data transmission system 100 may include a storage device configured to store data, instructions, and/or any other information. Additionally or alternatively, two or more components of the data transmission system 100 may be integrated into a single component. A component of the data transmission system 100 may be implemented on two or more sub-components.

FIG. 2 is a flowchart illustrating an exemplary process 200 for establishing a first communication link according to some embodiments of the present disclosure. In some embodiments, the process 200 may be performed via the data transmission systems 300, 400, 600A, or 600B.

In some embodiments, the signal converting device 11 may include one or more ADCs. Since clocks of the one or more ADCs are likely to be asynchronous, before establishing the first communication link, the clocks of the one or more ADCs need to be synchronized.

In 210, the signal processing device 12 may transmit a clock synchronization instruction to the signal converting device 11 via a second communication link between the signal converting device 11 and the signal processing device 12.

The clock synchronization instruction may be used to instruct the signal converting device 11 to perform clock synchronization on one or more ADCs of the signal converting device 11. In some embodiments, the clock synchronization instruction may be transmitted in the form of a clock signal, and the clock signal may be generated via a clock source of the signal processing device 12. In some embodiments, the clock synchronization instruction may be transmitted in the form of high-speed data, and the high-speed data may be generated via a data processor of the signal processing device 12.

The second communication link may be used to transmit the clock synchronization instruction. In some embodiments, the second communication link may be established between the clock source of the signal processing device 12 and a clock synchronizer of the signal converting device 11 (e.g., when the clock synchronization instruction may be transmitted in the form of a clock signal). In some embodiments, the second communication link may be established between the data processor of the signal processing device 12 and a configurator of the signal converting device 11 (e.g., when the clock synchronization instruction may be transmitted in the form of high-speed data).

In 220, in response to the clock synchronization instruction, the signal converting device 11 may perform clock synchronization on one or more ADCs.

In some embodiments, when the clock synchronization instruction may be transmitted in the form of a clock signal, the clock synchronizer of the signal converting device 11 may be configured to receive the clock signal via the second communication link and perform the clock synchronization on the one or more ADCs based on the clock signal. In some embodiments, when the clock synchronization instruction may be transmitted in the form of high-speed data, the configurator of the signal converting device 11 may receive the high-speed data via the second communication link, restore the clock signal from the high-speed data, and perform the clock synchronization on the one or more ADCs based on the clock signal.

In 230, the signal converting device 11 may transmit a connection request to the signal processing device 12 after the clock synchronization is achieved.

The connection request may be used to request the establishment of the first communication link. In some embodiments, the first communication link may be established between a data transmitter of the signal converting device 11 and a data receiver of the signal processing device 12.

In 240, the signal processing device 12 may generate and transmit a connection instruction to the signal converting device 11 in response to the connection request.

The connection instruction may be used to indicate that the signal processing device 12 is ready to establish the first communication link, and to prompt the signal converting device 11 to send link data the signal processing device 12. In some embodiments, the connection instruction may be a slow speed connection instruction.

In 250, in response to the connection instruction, the signal converting device 11 may generate link data and transmit the link data to the signal processing device 12.

In some embodiments, the signal converting device 11 may generate the link data based on information in the connection instruction. For example, the configurator of the signal converting device 11 may generate one or more synchronization signals in response to the connection instruction, and transmit the one or more synchronization signals to the one or more ADCs, the one or more ADCs may generate the link data in response to the one or more synchronization signals.

In 260, the signal processing device 12 may confirm whether the first communication link is established based on the link data.

In some embodiments, if the signal processing device 12 receives the link data, the signal processing device 12 may confirm that the first communication link is established.

In some embodiments, different data transmitted from the signal converting device 11 to the signal processing device 12 may include different identifiers. The signal processing device 12 may confirm whether the first communication link is established based on an identifier in the link data. For example, the connect request may include an identifier BC, the link data may include an identifier AD. If data received by the signal processing device 12 include the identifier AD, the signal processing device 12 may confirm that the first communication link is established. If data received by the signal processing device 12 include another identifier other than the identifier AD, the signal processing device 12 may confirm that the first communication link is not established.

In some embodiments, the signal processing device 12 may confirm whether the first communication link is established based on one or more parameters in the link data. Exemplary parameters may include a phase, a format, or the like, or any combination thereof. For example, the signal processing device 12 may determine whether each parameter in the link data has a target value. In response to determining that each parameter in the link data has the target value, the signal processing device 12 may confirm that the first communication link is established. In response to determining that values of one or more parameters in the link data are not the target values, the signal processing device 12 may confirm that the first communication link is not established. In some embodiments, the target value of each parameter may be preset previously by data transmission system 100. In some embodiments, the connect request may include the target value of each parameter.

In some embodiments, before the link data is transmitted to the signal processing device 12, the signal converting device 11 may continue to transmit initial data (e.g., the connection request) with the one or more parameters of initial values to the signal processing device 12. If the values of the one or more parameters (e.g., the phase) in the data received by the signal processing device 12 change, that is, the data received by the signal processing device 12 changes from the initial data to the link data, the signal processing device 12 may confirm that the first communication link is established. If the values of the one or more parameters in the data received by the signal processing device 12 do not change, that is, the data received by the signal processing device 12 changes is still the initial data, the signal processing device 12 may confirm that the first communication link is not established. Merly by way of example, if the value of the phase in the data received by the signal processing device 12 changes, the signal processing device 12 may confirm that the first communication link is established; If the value of the phase does not change, the signal processing device 12 may confirm that the first communication link is not established.

In some embodiments, if the signal processing device 12 may confirm that the first communication link is not established, the process 200 may be repeated, or the signal processing device 12 may further perform 240 to generate a new connection instruction, and operations 240-260 may be repeated.

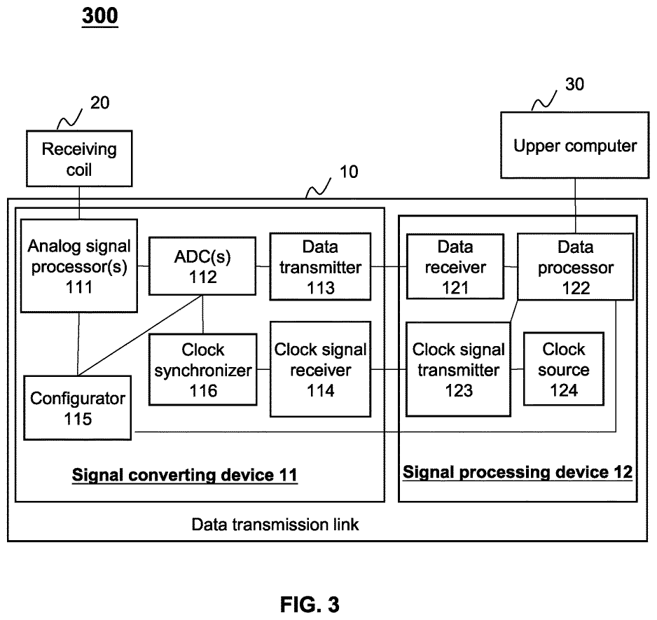

FIG. 3 is a schematic diagram illustrating an exemplary data transmission system 300 according to some embodiments of the present disclosure. The data transmission system 300 may be an example of the data transmission system 100 described in FIG. 1A. The data transmission system 300 may be used to implement the process 200 in FIG. 2.

As illustrated in FIG. 3, the signal converting device 11 may include one or more analog signal processors 111, one or more ADCs 112, a data transmitter 113, a clock signal receiver 114, a configurator 115, and a clock synchronizer 116. The signal processing device 12 may include a data receiver 121, a data processor 122, a clock signal transmitter 123, and a clock source 124.

Each analog signal processor 111 may be connect to one ADC 112 and the receiving coil 20. An analog signal processor 111 may be configured to generate preprocessed analog MR signals by preprocessing the analog MR signals received from the receiving coil 20, and transmit the preprocessed analog MR signals to the corresponding ADC 112.

In some embodiments, the one or more ADCs 112 may be further connected to the data transmitter 113. An ADC may be configured to transform the preprocessed analog MR signals received by from one analog signal processor 111 into digital MR signals, and the transmit the digital MR signals to the data transmitter 113. The ADCs may include a direct-conversion ADC, a successive-approximation ADC, a ramp-compare ADC, a Wilkinson ADC, an integrating ADC, a delta-encoded ADC, a pipeline ADC, a sigma-delta ADC, or the like, or a combination thereof. In some embodiments, the one or more ADCs 112 may include one or more single-channel ADCs, a multichannel ADC, or an ADC integrated with a digital signal processing function. The multichannel ADC may include multiple channels each of which is similar to one single-channel ADC. The ADC integrated with a digital signal processing function may perform some operations on MR signals. Exemplary operations may include a filter operation, a frequency conversion operation, an up and down conversion operation, an extraction operation, an interpolation operation, and a combination operation, or the like, or any combination thereof. In some embodiments, the ADC may process the MR signals using an AI algorithm such as a machine learning algorithm (e.g., a neural network algorithm, a deep learning algorithm). The one or more ADCs 112 may output data using a high-speed data transmission protocol such as a high-speed peripheral component interconnect express (PCIE) protocol, a JESD204B protocol, a LVDS protocol, a self-built high-speed protocol, etc.

The data transmitter 113 may be further connected to the data receiver 121, and configured to transmit data (e.g., the link data, the transmission data generated based on the digital MR signals) to the data receiver 121. The first communication link described elsewhere in the present disclosure may be achieved by a wired or wireless connection between the data transmitter 113 and the data receiver 121. The data transmitter 113 may include a high-frequency long-distance coaxial cable, an optic module, an up-conversion RF module with a frequency higher than an imaging frequency band, etc. The optic module may include a single-mode optic module or a multi-mode optic module. The optic module may connect an optical fiber or transmit space light. The up-conversion RF module may include an RF module with carrier waves of 800 MHz, an RF module with carrier waves of 1 GHz, an RF module with carrier waves of 2.4 GHz, an RF module with carrier waves of 5.1 GHz, an RF module with carrier waves of 5.8 GHz, an RF module with carrier waves of 10 GHz, an RF module with carrier waves of 24 GHz, an RF module with carrier waves of 60 GHz, an RF module with carrier waves of 67 GHz, an RF module with carrier waves of 70 GHz, an RF module with carrier waves of 120 GHz, etc.

The clock signal receiver 114 may be connected to the clock signal transmitter 123 and the clock synchronizer 116. The clock signal receiver 114 may be configured to receive a clock signal from the clock signal transmitter 123, and transmit the clock signal to the clock synchronizer 116.

The clock synchronizer 116 may be further connected to the one or more ADCs 112. The clock synchronizer 116 may be configured to receive the clock signal and perform the clock synchronization on the one or more ADCs based on the clock signal. In some embodiments, the clock synchronizer 116 may be a clock fanout buffer.

In some embodiments, the configurator 115 may be connected to the data processor 122 and the one or more ADCs 112. The configurator 115 may be configured to receive a connection instruction from the data processor 122, generate one or more synchronization signals in response to the connection instruction, and transmit the one or more synchronization signals to the one or more ADCs 112. The one or more ADCs may be configured to generate link data in response to the one or more synchronization signals. In some embodiments, the configurator 115 may be further connected the one or more analog signal processors 111. The configurator 115 may be configured to receive a signal preprocessing instruction from the signal processing device 12, and transmit the signal preprocessing instruction to the one or more analog signal processors 111 to direct to the one or more analog signal processors 111 to generate the preprocessed analog MR signals. In some embodiments, the configurator 115 may be a complex programmable logic device (CPLD).

The data receiver 121 may be configured to receive data (e.g., the link data, the transmission data generated based on the digital MR signals) transmitted from the data transmitter 113. The data receiver 121 may be further connected to the data processor 122, and configured to transmit data the to the data processor 122. In some embodiments, the type of the data receiver 121 may be the same as the type of the data transmitter 113. For example, the data receiver 121 may include a high-frequency long-distance coaxial cable, an optic module, an up-conversion RF module with a frequency higher than an imaging frequency band, etc. The optic module may include a single-mode optic module or a multi-mode optic module.

The data processor 122 may be further connected to the upper computer 30, and configured to process data received from the data receiver 121 and the upper computer 30. For example, the data processor 122 may resolve the digital MR signals from the transmission data received from the data receiver 121. In some embodiments, the data processor 122 may perform one or more operations according to an instruction received from the upper computer 30. For example, during processing digital MR signals, the upper computer 30 may send a modification instruction to the data processor 122 in a wireless manner. In response to the modification instruction, the data processor 122 may modify parameters during the processing and transmit the processed digital MR signals to the upper computer 30. After receiving the processed digital MR signals, the upper computer 30 may further process the digital MR signals to generate an MR image. As another example, the upper computer 30 may send a data filtering instruction to the data processor 122. In response to the data filtering instruction, the data processor 122 may discard part of the digital MR signals and upload the remaining digital MR signals to the upper computer 30. In this way, the data processor 122 does not need to control the status of the signal converting device 11 in real time, simplifying communication links for communication and saving communication media. As still another example, the upper computer 30 may send an operation modification instruction to the one or more analog signal processors 111 through the data processor 122. After receiving the operation modification instruction, the one or more analog signal processors 111 may adjust an amplifying operation, a filtering operation, a mixing frequency operation, etc.

In some embodiments, the data processor 122 may include a field programmable gate array (FPGA), a CPLD, a microcontroller unit (MCU), a system-on-a-chip (SOC), etc. The data processor 122 may have a high-speed data interface and use a high-speed data transmission protocol compatible with the protocol of the one or more ADCs 112. Exemplary high-speed data transmission protocols may include a PCIe protocol, a JESD204B protocol, a LVDS protocol, a self-built high-speed protocol, etc.

The clock signal transmitter 123 may be further connected to the clock source 124. The clock source 124 may be configured to generate a clock signal, and transmit the clock signal to the clock signal transmitter 123. In some embodiments, the clock signal transmitter 123 may be further connected to the data processor 122, and configured to transmit data from the data processor 122 to the clock signal receiver 114.

In some embodiments, the first communication link described in FIG. 2 may be established based on a clock signal, that is, the clock synchronization instruction described in FIG. 2 may be transmitted in the form of the clock signal. For illustration purposes, the establishment of the first communication link based on a clock signal is described hereinafter.

Specifically, the clock source 124 may generate the clock signal. The second communication link may be established between the clock source 124 and the clock synchronizer 116, the clock synchronizer 116 may receive the clock signal via the second communication link and perform the clock synchronization on the one or more ADCs 112 based on the clock signal. In some embodiments, the second communication link may be achieved by a wired cable connecting the clock source 124 and the clock synchronizer 116. In some embodiments, as shown in FIG. 3, the second communication link may be achieved by a wired or wireless connection between the clock signal transmitter 123 and the clock signal receiver 114. For example, the clock signal transmitter 123 and the clock signal receiver 114 may be connected by a wired cable. As another example, the clock signal transmitter 123 is a laser transmitting module, the clock signal receiver 114 is a laser receiving module, and the clock signal transmitter 123 and the clock signal receiver 114 may be wirelessly connected.

In some embodiments, to perform the clock synchronization on the one or more ADCs 112, for each of the one or more ADCs, the clock synchronizer 116 may generate a second clock signal (also referred to as a stable clock signal) corresponding to the ADC based on the clock signal. Further, the clock synchronizer 116 may transmit the second clock signal corresponding to the ADC to the ADC for performing the clock synchronization. The second clock signals corresponding to the one or more ADCs may be from the same clock origin. In some embodiments, the rising edges of the second clock signals are strictly aligned with the original clock signal, and phases of the second clock signals may be the same.

It should be noted that before the second clock signals are generated, the signal converting device 11 needs to initialize the clock synchronizer 116 and each ADC 112 with preset parameters. In some embodiments, after the signal converting device 11 is powered on, the initialization of the clock synchronizer 116 and each ADC 112 may be performed automatically.

Further, the signal converting device 11 (e.g., the data transmitter 113) may transmit a connection request to the signal processing device 12 (e.g., the data receiver 121) after the clock synchronization is achieved. For example, the connection request may be a digital RF signal. When the data transmitter 113 is a multi-mode optic module, the digital RF signal (i.e., the connection request) output by the ADC may be converted into an optical signal, and transmitted to the data receiver 121.

Then, the signal processing device 12 (e.g., the data processor 122) may transmit a connection instruction to the configurator 115 in response to the connection request. The configurator 115 may receive the connection instruction from the signal processing device 12 via a third communication link between the signal converting device 11 and the signal processing device 12. In some embodiments, the third communication link is established between the configurator 115 and the data processor 122. Similar to the second communication link, the third communication link may be achieved by a wired or wireless connection between the data processor 122 and the configurator 115. In some embodiments, the data processor 122 may be connected to the clock signal transmitter 123 and the configurator 115 may be connected to the clock signal receiver 114. The data processor 122 may transmit the connection instruction to the configurator 115 via the clock signal transmitter 123 and the clock signal receiver 114. In this case, the second communication link and the third communication link may be the same.

The configurator 115 may generate one or more synchronization signals in response to the connection instruction, and transmit the one or more synchronization signals to the one or more ADCs 112. In some embodiments, for each ADC 112, the configurator 115 may generate one synchronization signal corresponding to the ADC, and transmit the synchronization signal to the ADC.

Then, the one or more ADCs 112 may generate link data in response to the one or more synchronization signals. In some embodiments, the one or more ADCs 112 may obtain initial data, adjust the format and/or the phase of the initial data, and transform the adjusted initial data into the link data based on a digital protocol of the data processor 122. The link data may be transmitted to the data processor 122 via the transmitter 113 and the data receiver 121. In some embodiments, the one or more ADCs 112 may adjust the format and/or the phase of the initial data based on the digital protocol of the data processor 122, and designate the adjusted initial data as the link data. The digital protocol specifies requirements that features (e.g., (a format, an encoding manner, etc.) of the data received by the data processor 122 need to satisfy. For example, the digital protocol may be a high-speed low-voltage differential signaling (LVDS) protocol. In this case, the data transmitter 113 may be the multi-mode optic module. As another example, the digital protocol may be the JESD204B protocol. In this case, the data transmitter 113 may be a millimeter wave transmitter.

In some embodiments, the initial data may be data directly generated by the one or more ADCs, such as special character codes (e.g., k, h, j, etc.). For example, the initial data is k, the ADC(s) may adjust the k to j. In some embodiments, the initial data may be target digital MR signals converted from a portion of the analog MR signals. For example, the portion of the analog MR signals are collected at a preset time interval after the MR scan starts. In some embodiments, the one or more ADCs may directly designate the adjusted initial data as the link data. In some embodiments, the one or more ADCs may transform the adjusted initial data into the link data. Specifically, the adjusted initial data may be encoded according to the digital protocol and converted into link data that conforms to the digital protocol.

In some embodiments, each analog signal processor 111 may be connected to the receiving coil 20, the configurator 115, and one ADC 112. The configurator 115 may receive a signal preprocessing instruction including one or more operating parameter values of the one or more analog signal processors 111 from the signal processing device 12, and transmit the one or more operating parameter values to the one or more analog signal processors 111. Exemplary operating parameter values may include an operating parameter value relating to an amplifying operation, an operating parameter value relating to a filtering operation, an operating parameter value relating to a mixing frequency operation, an operating parameter value relating to a gain operation, or the like, or any combination thereof.

Further, the one or more analog signal processors 111 may generate preprocessed analog MR signals by preprocessing the portion of the analog MR signals based on the one or more operating parameter values, and transmit the preprocessed analog MR signals to the one or more ADCs 112. Exemplary preprocessing operations may include an amplifying operation, a filtering operation, a mixing frequency operation, a gaining operation, or the like, or any combination thereof. To convert the portion of the analog MR signals into the target digital MR signals, the one or more ADCs 112 may receive the preprocessed analog MR signals from the one or more analog signal processors 111 and convert the preprocessed analog MR signals into the target digital MR signals.

In some embodiments, each analog signal processor 111 may include an analog signal filter and/or an analog signal gainer, operating parameters of the analog signal filter and/or the analog signal gainer may be adjustable. For example, the operating parameters of the analog signal filter may indicate whether the filtering operation needs to be performed on the analog MR signals. The operating parameters of the analog signal gainer may indicate whether the gaining operation needs to be performed on the analog MR signals. In some embodiments, the operating parameters of the analog signal gainer may indicate whether a relatively low gain or a relatively high gain needs to be performed on the analog MR signals. In some embodiments, the one or more operating parameter values may be determined at least based on the number of nuclides imaged in the MR scan.

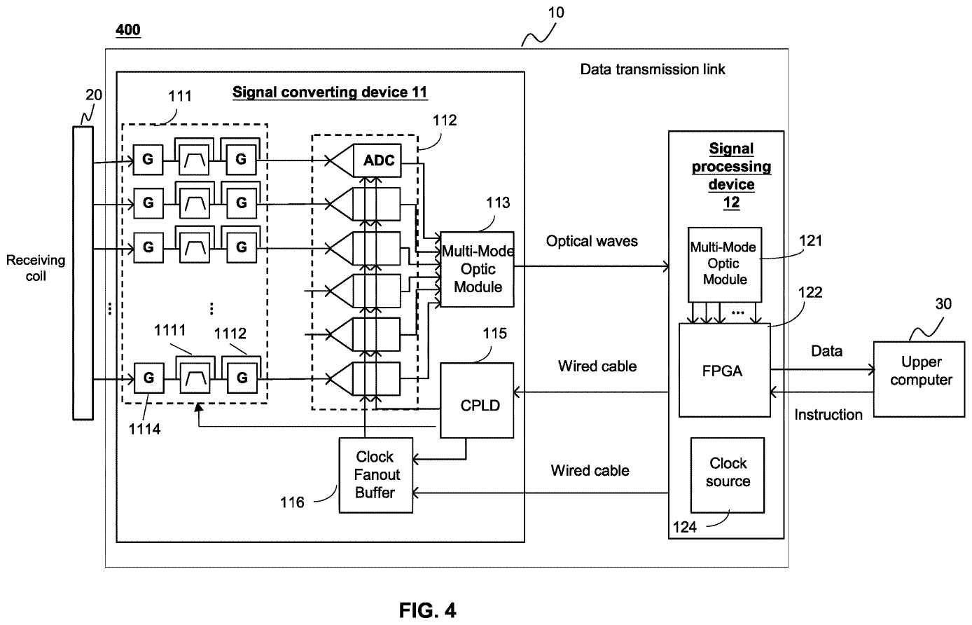

FIG. 4 is a schematic diagram illustrating an exemplary data transmission system 400 according to some embodiments of the present disclosure. The data transmission system 400 may be an example of the data transmission system 300 described in FIG. 3. As shown in FIG. 4, the data transmitter 113 is a multi-mode optic module, the configurator 115 is a CPLD, the clock synchronizer 116 is a clock fanout buffer, the data receiver 121 is a multi-mode optic module, and the data processor 122 is FPGA. The multi-mode optic module 113 transmits data to the multi-mode optic module 121 via optical waves. The FPGA 122 transmits an instruction to the CPLD 115 via a wired cable. The clock source 124 transmits a clock signal to the clock fanout buffer 116 via a wired cable. The FPGA 122 transmits data to the upper computer 30, and the upper computer 30 sends an instruction to the FPGA 122. Each analog signal processor 111 includes an analog signal filter 1111, an analog signal gainer 1112, and an analog signal gainer 1114. The analog signal filter 1111 is connected to the analog signal gainer 1112 and the analog signal gainer 1114, respectively. The operating parameters of the analog signal filter 1111 and the analog signal gainer 1112 may be adjustable. For example, the operating parameters of the analog signal filter 1111 may indicate whether the filtering operation needs to be performed on the analog MR signals. Operating parameters of the analog signal gainer 1112 may indicate whether the gaining operation needs to be performed on the analog MR signals or whether a low gain or a high gain needs to be performed on the analog MR signals.

Specifically, the upper computer 30 may generate a signal preprocessing instruction and send the signal preprocessing instruction to the FPGA 122. The signal preprocessing instruction includes operating parameter values of the analog signal filter 1111 and the analog signal gainer 1112 of each analog signal processor 111. The CPLD 115 may receive the signal preprocessing instruction from the FPGA 122 via the wired cable, and transmit the operating parameter values to the analog signal processors 111. In some embodiments, the FPGA 122 may determine the operating parameter values according to the number of nuclides imaged in the MR scan. For example, when multiple nuclides simultaneously are received, the analog signal filter 1111 does not need to perform the filtering operation.

Further, the analog signal processors 111 may generate preprocessed analog MR signals by preprocessing the portion of the analog MR signals based on the operating parameter values, and transmit the preprocessed analog MR signals to the one or more ADCs 112. The one or more ADCs 112 may receive the preprocessed analog MR signals from the analog signal processors 111 and convert the preprocessed analog MR signals into the target digital MR signals.

In some embodiments, after the first communication link is established, the signal converting device 11 and the signal processing device 12 may maintain a wired communication state. The FPGA 122 may discard the invalid digital MR signals and send the remaining digital MR signals according to an instruction of the upper computer 30 to the upper computer 30. In some embodiments, after the first communication link is established and before using the first communication link to transmit MR signals, the upper computer 30 may send an instruction to the FPGA 122. After the instruction is received, the FPGA may parse and encapsulate the instruction, and issue a secondary instruction to the CPLD 115. After receiving the secondary instruction, the CPLD 115 may analyze the secondary instruction and control the one or more analog signal processors 111 to perform preprocessing operations (e.g., the amplifying operation, the filtering operation, the mixing frequency operation, etc.) on the analog MR signals collected by the receiver coil 20.

In the process of receiving analog MR signals using the first communication link, the LVDS protocol may be used. It should be understood that the LVDS protocol is an exemplary protocol provided for illustration purposes. The one or more analog signal processors 111 may perform preprocessing operations (e.g., the amplifying operation, the filtering operation, the mixing frequency operation, etc.) on the analog MR signals collected by the receiver coil 20. The preprocessed analog MR signals may be transmitted to the one or more ADCs 112, and the one or more ADC 112 may convert the analog MR signals into LVDS protocol digital signals. The multi-mode optic module 113 may convert the LVDS protocol digital signals into multi-mode optical signals and transmit the multi-mode optical signals to the signal processing device 12 via multi-mode optical fiber. The multi-mode optic module 121 in the signal processing device 12 may re-convert the multi-mode optical signals into LVDS protocol digital signals and transmit the LVDS protocol digital signals to a high-speed digital interface of the FPGA 122. After the FPGA 122 parses the digital MR signals according to the LVDS protocol, the FPGA 122 may perform operations (e.g., a filter operation, a frequency conversion operation, a compression and package operation) on the digital MR signals, and then upload the processed digital MR signals to the upper computer 30. The upper computer 30 may perform data processing and generate an MR image. It should be noted that all ADCs 112 use the clock signal generated by the same clock source 124, which can be transmitted through the wired cable.

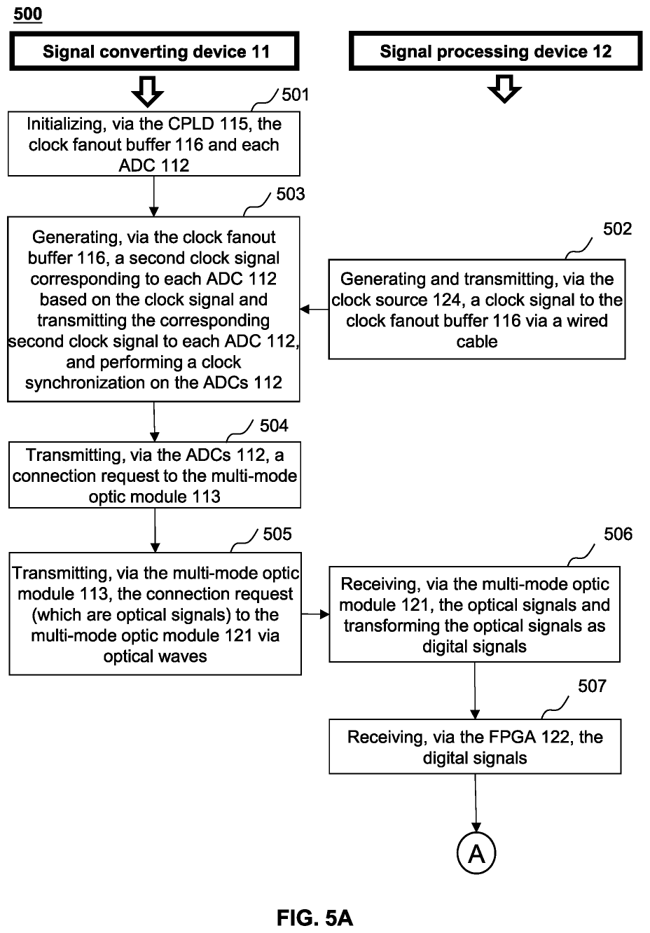

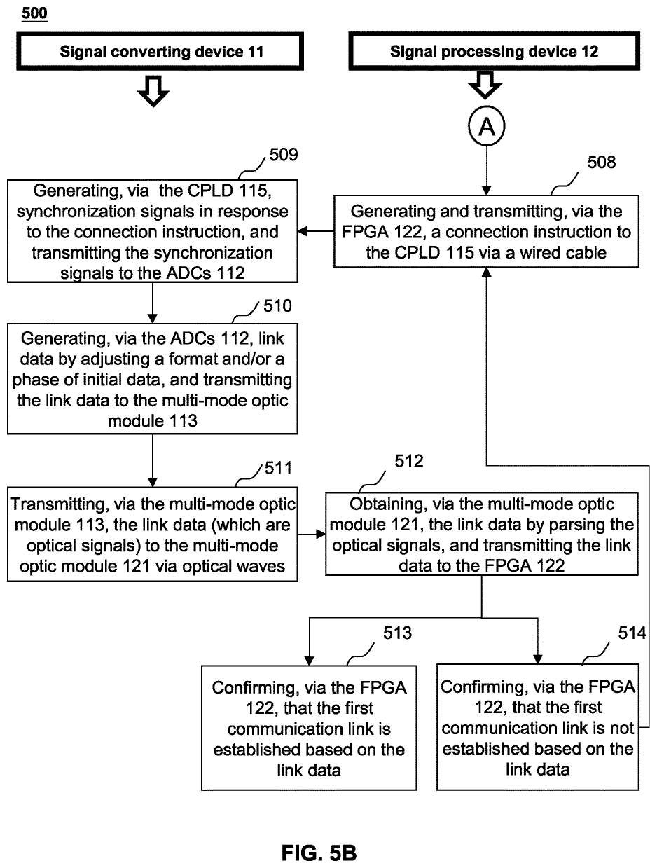

FIGS. 5A and 5B are flowcharts illustrating an exemplary process 500 for establishing a first communication link according to some embodiments of the present disclosure. The process 500 may be performed by the data transmission system 400 in FIG. 4.

In 501, the CPLD 115 may initialize the clock fanout buffer 116 and each ADC 112.

In 502, the clock source 124 may generate and transmit a clock signal to the clock fanout buffer 116 via a wired cable.

In 503, the clock fanout buffer 116 may generate a second clock signal corresponding to each ADC 112 based on the clock signal and transmit the corresponding second clock signal to each ADC 112 to perform a clock synchronization on the ADCs 112.

In 504, the ADCs 112 may transmit a connection request to the multi-mode optic module 113.

In 505, the multi-mode optic module 113 may transmit the connection request (which are optical signals) to the multi-mode optic module 121 via optical waves.

In 506, the multi-mode optic module 121 may receive the optical signals and transform the optical signals as digital signals.

In 507, the FPGA 122 may receive the digital signals.

In 508, the FPGA 122 may generate and transmit a connection instruction to the CPLD 115 via a wired cable.

In 509, the CPLD 115 may generate synchronization signals in response to the connection instruction, and transmit the synchronization signals to the ADCs 112.

In 510, the ADCs 112 may generate link data by adjusting a format and/or a phase of initial data, and transmit the link data to the multi-mode optic module 113.

In 511, the multi-mode optic module 113 may transmit the link data (which are optical signals) to the multi-mode optic module 121 via optical waves.

In 512, the multi-mode optic module 121 may obtain the link data by parsing the optical signals, and transmit the link data to the FPGA 122.

Then, the FPGA 122 may perform operation 513 or operation 514. In 513, the FPGA 122 may confirm that the first communication link is established based on the link data. In 514, the FPGA 122 may confirm that the first communication link is not established based on the link data. If operation 514 is performed, the FPGA 122 may further perform 508 to generate a new connection instruction and transmit the new connection instruction to the CPLD 115.

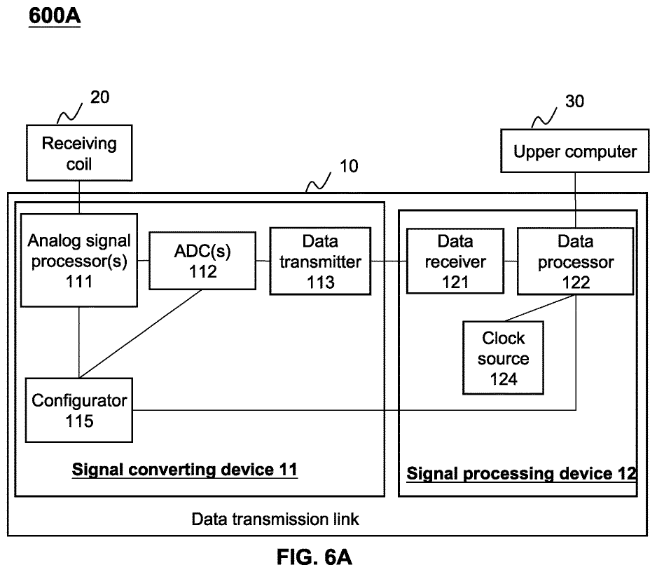

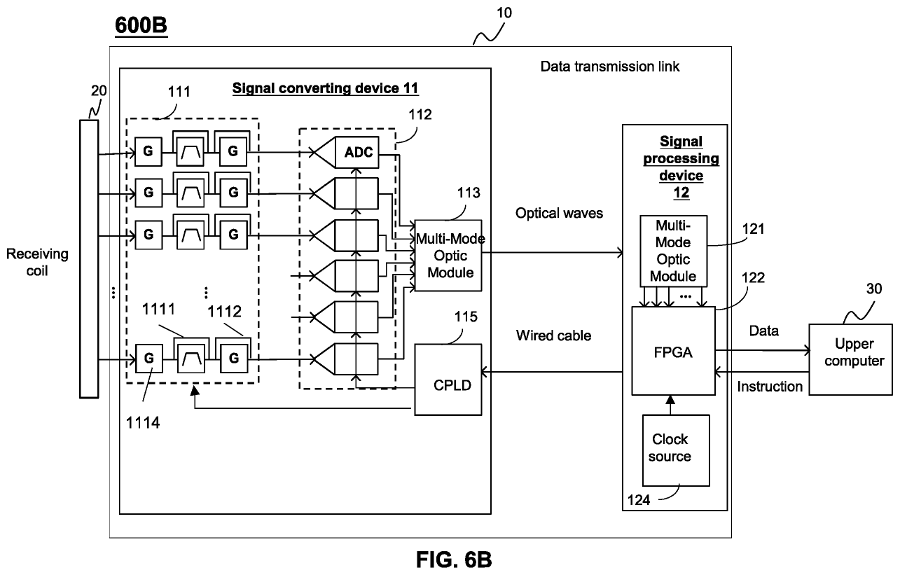

FIG. 6A is a schematic diagram illustrating an exemplary data transmission system 600A according to some embodiments of the present disclosure. The data transmission system 600A is an exemplary embodiment of the data transmission system 100 as described in connection with FIG. 1A.

As illustrated in FIG. 6A, the data transmission system 600A is similar to the data transmission system 300 as described in connection with FIG. 3, except that certain features. The clock signal transmitter 123, the clock signal receiver 114, and the clock synchronizer 116 are omitted from the data transmission system 600A, and the clock source 124 is connected to the data processor 122.

When establishing the first communication link in the data transmission system 600A, the clock synchronization instruction may be transmitted in the form of high-speed data. Specifically, the clock source 124 may be configured to generate a clock signal, and the data processor 122 may be configured to generate the high-speed data based on the clock signal. In some embodiments, the connection instruction may be also transmitted in the form of high-speed data.

The second communication link may be established between the data processor 122 and the configurator 115. The configurator 115 may be configured to receive the high-speed data via the second communication link, restore the clock signal from the high-speed data, and perform the clock synchronization on the one or more ADCs 112 based on the clock signal.

In some embodiments, the signal converting device 11 may further comprise one or more analog signal processors 111. Each analog signal processor 111 may be connected to the receiving coil 20, the configurator 115, and one of the one or more ADCs 112. The high-speed data may further include one or more operating parameter values of the one or more analog signal processors 111. The configurator 115 may be further configured to restore the one or more operating parameter values from the high-speed data, and transmit the one or more operating parameter values to the one or more analog signal processors 111.

FIG. 6B is a schematic diagram illustrating an exemplary data transmission system 600B according to some embodiments of the present disclosure. The data transmission system 600B is an exemplary embodiment of the data transmission system 600A. As shown in FIG. 6B, the data transmitter 113 is a multi-mode optic module, the configurator 115 is a CPLD, the data receiver 121 is a multi-mode optic module, and the data processor 122 is FPGA. The analog signal processor(s) 111 and the ADC(s) 112 are similar to those in FIG. 4, the descriptions of which are not repeated.

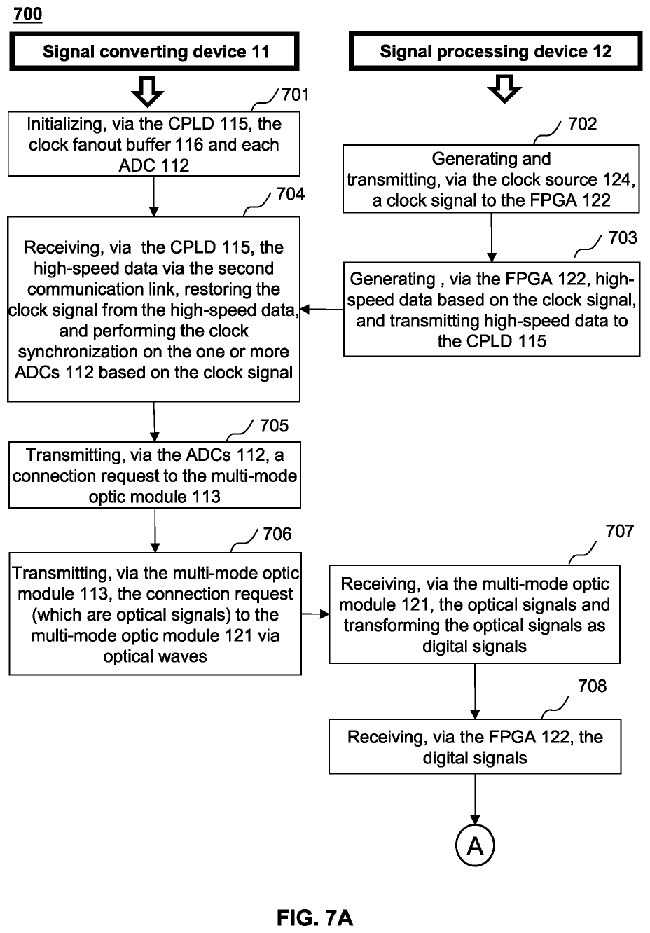

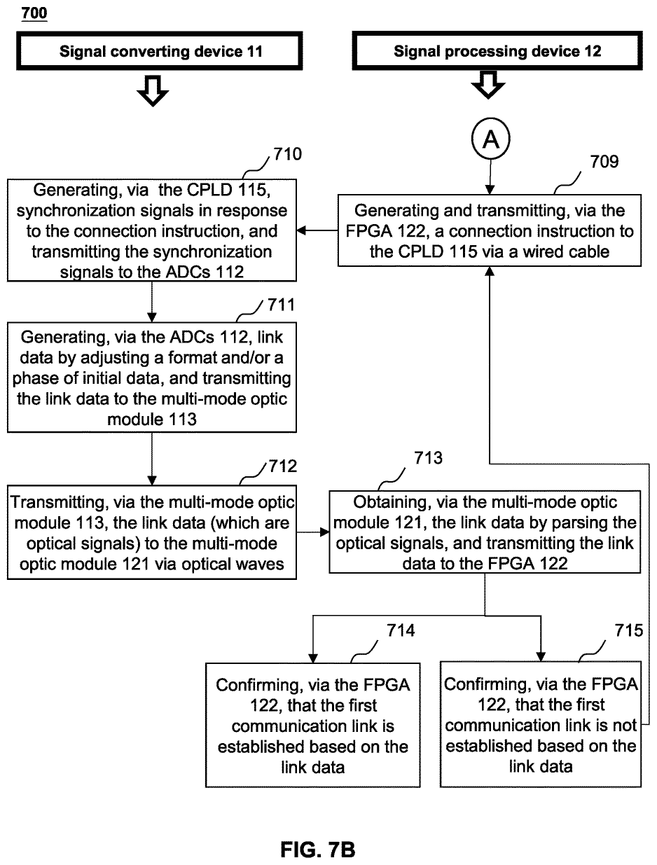

FIGS. 7A and 7B are flowcharts illustrating an exemplary process 700 for establishing a first communication link according to some embodiments of the present disclosure. The process 700 may be performed by the data transmission system 600B in FIG. 6B.

In 701, the CPLD 115 may initialize each ADC 112.

In 702, the clock source 124 generate and transmit a clock signal to the FPGA 122.

In 703, the FPGA 122 may generate high-speed data based on the clock signal, and transmit high-speed data to the CPLD 115.

In 704, the CPLD 115 may receive the high-speed data via the second communication link, restore the clock signal from the high-speed data, and perform the clock synchronization on the one or more ADCs 112 based on the clock signal.

In 705, the ADCs 112 may transmit a connection request to the multi-mode optic module 113.

In 706, the multi-mode optic module 113 may transmit the connection request (which are optical signals) to the multi-mode optic module 121 via optical waves.

In 707, the multi-mode optic module 121 may receive the optical signals and transform the optical signals as digital signals.

In 708, the FPGA 122 may receive the digital signals.

In 709, the FPGA 122 may generate and transmit a connection instruction to the CPLD 115 via a wired cable. In some embodiments, the connection instruction may be transmitted in the form of high-speed data.

In 710, the CPLD 115 may generate synchronization signals in response to the connection instruction, and transmit the synchronization signals to the ADCs 112.

In 711, the ADCs 112 may generate link data by adjusting a format and/or a phase of initial data, and transmit the link data to the multi-mode optic module 113.

In 712, the multi-mode optic module 113 may transmit the link data (which are optical signals) to the multi-mode optic module 121 via optical waves.

In 713, the multi-mode optic module 121 may obtain the link data by parsing the optical signals, and transmit the link data to the FPGA 122.

Then, the FPGA 122 may perform operation 714 or operation 715. In 714, the FPGA 122 may confirm that the first communication link is established based on the link data. In 715, the FPGA 122 may confirm that the first communication link is not established based on the link data. If operation 715 is performed, the FPGA 122 may further perform 709 to generate a new connection instruction and transmit the new connection instruction to the CPLD 115.

FIG. 8 is a flowchart illustrating an exemplary process 800 for establishing a first communication link according to some embodiments of the present disclosure. In some embodiments, the process 800 may be performed via the data transmission systems 900, 1000, 1200A, or 1200B. Different from the process 200 in which the signal processing device 12 transmits a connection instruction to the signal converting device 11, in the process 800, the signal converting device 11 and the signal processing device 12 need to generate connection instructions, respectively.

As described elsewhere in the present disclosure, the signal converting device 11 may include one or more ADCs. Since clocks of the one or more ADCs are likely to be asynchronous, before establishing the first communication link, the clocks of the one or more ADCs need to be synchronized.

In 810, the signal processing device 12 may transmit a clock synchronization instruction to the signal converting device 11 via a second communication link between the signal converting device 11 and the signal processing device 12.

In some embodiments, the operation 810 may be performed in a same or similar manner as the operation 210. For example, in some embodiments, the clock synchronization instruction may be transmitted in the form of a clock signal, and the clock signal may be generated via a clock source of the signal processing device 12. In some embodiments, the clock synchronization instruction may be transmitted in the form of high-speed data, and the high-speed data may be generated via a data processor of the signal processing device 12. In some embodiments, the second communication link may be established between the clock source of the signal processing device 12 and a clock synchronizer of the signal converting device 11. In some embodiments, the second communication link may be established between the data processor of the signal processing device 12 and a configurator of the signal converting device 11.

In 820, in response to the clock synchronization signal, the signal converting device 11 may perform clock synchronization on one or more ADCs and start timing in response to the clock synchronization instruction.

In some embodiments, the clock synchronizer of the signal converting device 11 may be configured to receive the clock signal from a clock source of the via the second communication link, perform the clock synchronization on the one or more ADCs based on the clock signal, and transmit a clock-locking signal to the configurator. The configurator may be configured to start timing in response to the clock-locking signal.

In 830, the signal converting device 11 may transmit a connection request to the signal processing device 12 after the clock synchronization is achieved.

The connection request may be used to request the establishment of the first communication link. In some embodiments, the first communication link may be established between a data transmitter of the signal converting device 11 and a data receiver of the signal processing device 12.

In 840, the signal converting device 11 may generate a connection instruction (also referred to as a first connection instruction) after a first preset period.

The first connection instruction may be used to indicate the signal converting device 11 to send link data the signal processing device 12. In some embodiments, the first connection instruction may be a slow speed connection instruction. The first preset period may be set manually by a user (e.g., an engineer) according to an experience value or a default setting of the data transmission system 100, such as 10 seconds, 20 seconds, 30 seconds, etc. In some embodiments, the first connection instruction may be the same as or similar to the connection instruction described in operation 508 or operation 709.

In 850, the signal processing device 12 may start timing in response to the connection request, and generate a second connection instruction corresponding to the first connection instruction after a second preset period.

The second connection instruction may be used to indicate that the signal processing device 12 is ready to establish the first communication link with the signal converting device 11. The second preset period may be set manually by a user (e.g., an engineer) according to an experience value or a default setting of the data transmission system 100, such as 10 seconds, 20 seconds, 30 seconds, etc. In some embodiments, the second preset period may be shorter than the first preset period. For example, the second preset period is 10 seconds, and the first preset period is 20 seconds.

In 860, in response to the first connection instruction, the signal converting device 11 may generate link data and transmit the link data to the signal processing device 12.

In some embodiments, the operation 860 may be performed in a same or similar manner as the operation 250. For example, the configurator of the signal converting device 11 may be configured to transmit the first connection instruction to each of the one or more ADCs, and the one or more ADCs may be configured to generate the link data in response to the first connection instruction.

In 870, the signal processing device 12 may confirm whether the first communication link is established based on the link data.

In some embodiments, the operation 870 may be performed in a same or similar manner as the operation 260.

In some embodiments, if the signal processing device 12 determines that the first communication link is not established, the process 800 may be repeated. For example, if the signal processing device 12 determines that the first communication link is not established, the signal processing device 12 may shut down the clock signal transmitter, the clock signal generated by the clock source is not transmitted to the signal converting device 11. After a preset interval of time, the signal processing device 12 may turn on the clock signal transmitter, and the process 800 may be repeated.

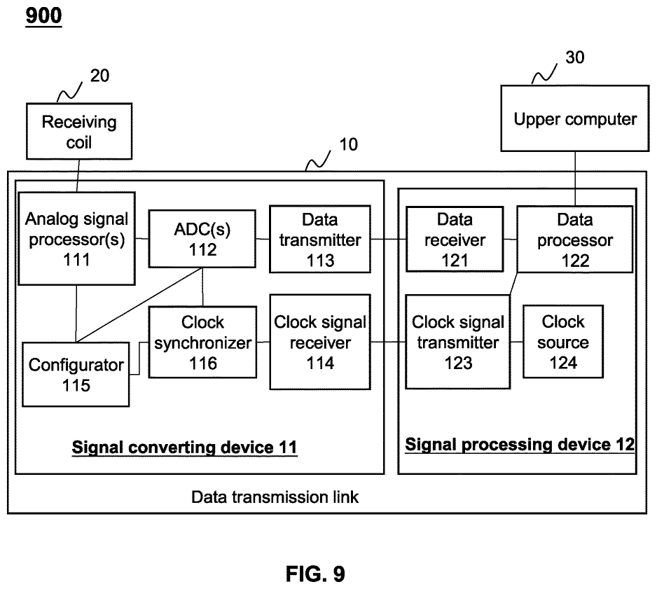

FIG. 9 is a schematic diagram illustrating an exemplary data transmission system 900 according to some embodiments of the present disclosure. The data transmission system 900 may be an example of the data transmission system 100 described in FIG. 1A. The data transmission system 900 may be used to implement the process 800 in FIG. 8.

As illustrated in FIG. 9, the data transmission system 900 may be similar to the data transmission system 300 as described in connection with FIG. 3, except that, in the data transmission system 900, the clock synchronizer 116 is connected to the configurator 115.

In some embodiments, the configurator 115 may be a microcontroller unit (MCU). In some embodiments, the clock synchronizer 116 may be a phase-locked loop. In some embodiments, the data processor 122 may be a SOC. In some embodiments, the clock signal transmitter 123 may be a laser emission module, and the clock signal receiver 114 may be a laser receiving module. In some embodiments, the data transmitter 113 may be a millimeter wave transmission module, and the data receiver 121 may be a millimeter wave receiving module.

In some embodiments, in order to establish the first communication link in the data transmission system 900, the clock source 124 may generate a clock signal. The second communication link may be established between the clock source 124 and the clock synchronizer 116, for example, via the clock signal transmitter 123 and the clock signal receiver 114 as shown in FIG. 9. The clock synchronizer 116 may receive the clock signal from the clock source 124 via the second communication link, perform the clock synchronization on the one or more ADCs 112 based on the clock signal, and transmit a clock-locking signal to the configurator 115. More descriptions regarding the second communication link and the performing of the clock synchronization on the one or more ADCs 112 based on the clock signal may be found elsewhere in the present disclosure (e.g., FIG. 3 and the descriptions thereof).

Further, the configurator 115 may start timing in response to the clock-locking signal and generate a first connection instruction after a first preset period. The first connection instruction may be used to instruct the signal converting device 11 to send link data the signal processing device 12.

The configurator 115 may transmit the first connection instruction to each of the one or more ADCs 112. Further, the one or more ADCs 112 may generate the link data in response to the first connection instruction. Specifically, for each ADC 112, the configurator 115 may designate the first connection instruction as one synchronization signal corresponding to the ADC, and transmit the synchronization signal to the ADC. Then, the one or more ADCs 112 may generate link data in response to the one or more synchronization signals. In some embodiments, the one or more ADCs 112 may generate the link data in a similar manner as how the one or more ADCs 112 generate the link data based on the one or more synchronization signals described in FIG. 3, and the descriptions of which are not repeated here.

In some embodiments, the one or more analog signal processors 111 may receive the analog MR signals from receiver coil 20 and generate preprocessed analog MR signals by preprocessing the analog MR signals based on one or more operating parameter values, and transmit the preprocessed analog MR signals to the one or more ADCs 112. Exemplary operating parameter values may include an operating parameter value relating to an amplifying operation, an operating parameter value relating to a filtering operation, an operating parameter value relating to a mixing frequency operation, an operating parameter value relating to a gain operation, or the like, or any combination thereof. Exemplary preprocessing operations may include an amplifying operation, a filtering operation, a mixing frequency operation, a gaining operation, or the like, or any combination thereof.

In some embodiments, each analog signal processor 111 may further include an analog signal filter and/or an analog signal gainer. Operating parameters of the analog signal filter and/or the analog signal gainer may be non-adjustable. For example, the operating parameters of the analog signal filter may indicate that the filtering operation needs to be performed on the analog MR signals or that the filtering operation does not need to be performed on the analog MR signals. The operating parameters of the analog signal gainer may indicate that the gaining operation needs to be performed on the analog MR signals or that the gaining operation does not need to be performed on the analog MR signals. In some embodiments, the operating parameters of the analog signal gainer may indicate that a relatively low gain needs to be performed on the analog MR signals or that a relatively high gain needs to be performed on the analog MR signals.

In some embodiments, the clock synchronizer 116 may be a phase-locked loop, and the one or more ADCs 112 may include one multichannel ADC. Each analog signal processor 111 may include a frequency mixer and be connected to the receiving coil 20, the phase-locked loop 116, and the multichannel ADC 112. The phase-locked loop may be configured to adjust a mixing frequency of the frequency mixer of each analog signal processor 111 such that the frequency of preprocessed analog MR signals generated by each analog signal processor 111 is within a preset frequency range corresponding to the multichannel ADC. For example, if the frequency of signals that the multichannel ADC 112 can receive is 20M and the frequency of signals input into the frequency mixer of an analog signal processor 111 is 120M, the phase-locked loop may adjust the mixing frequency of the frequency mixer of the analog signal processor 111 by 100M such that the frequency of preprocessed analog MR signals generated by the analog signal processor 111 is 20M.

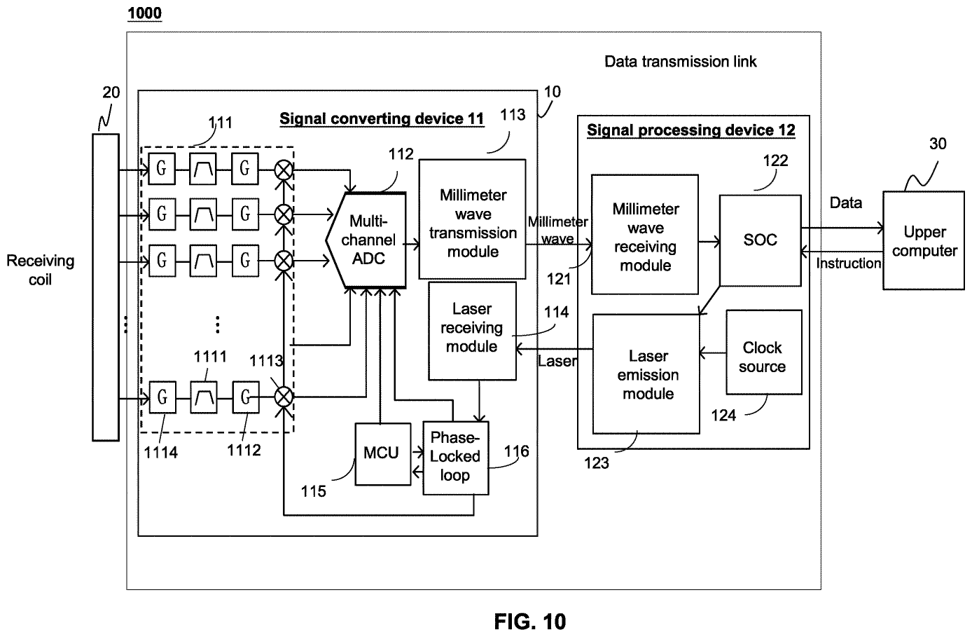

FIG. 10 is a schematic diagram illustrating an exemplary data transmission system 1000 according to some embodiments of the present disclosure. The data transmission system 1000 may be an example of the data transmission system 900 described in FIG. 9.

As shown in FIG. 10, the one or more ADCs 112 includes one multichannel ADC, the data transmitter 113 is a millimeter wave transmission module, the clock signal receiver 114 is a laser receiving module, the configurator 115 is an MCU, the clock synchronizer 116 is a phase-locked loop, the data receiver 121 is a millimeter wave receiving module, the data processor 122 is a SOC, and the clock signal transmitter 123 is a laser emission module. The millimeter wave receiving module 113 transmits data to the millimeter wave receiving module 121 via millimeter waves. The laser emission module 123 transmits data to the laser receiving module 114 via laser. Each analog signal processor 111 includes an analog signal filter 1111, an analog signal gainer 1112, an analog signal gainer 1114, and a frequency mixer 1113. The analog signal filter 1111 is connected to the analog signal gainer 1112 and the analog signal gainer 1114, respectively. Each frequency mixer 1113 is connected to the analog signal gainer 1112, the phase-locked loop 116, and the multichannel ADC 112, respectively. The phase-locked loop 116 may adjust a mixing frequency of the frequency mixer 1113 of each analog signal processor 111 such that the frequency of preprocessed analog MR signals generated by each analog signal processor 111 is within a preset frequency range corresponding to the multichannel ADC 112. The operating parameters of the analog signal filter 1111, the analog signal gainer 1112, and the analog signal gainer 1114 are non-adjustable.

Specifically, after the signal converting device 11 is powered on, the MCU 115 may initialize the phase-locked loop 116 and the multichannel ADC 112. At this time, the laser emission module 123 may wirelessly transmit the clock signal generated by the clock source 124 to the laser receiving module 114 via laser. Further, the phase-locked loop 116 may receive the clock signal from the laser receiving module 114, perform the clock synchronization on the multichannel ADC 112 based on the clock signal, and transmit a clock-locking signal to the MCU 115, to start establishing the first communication link.

A JESD204B protocol is described as an example. On the one hand, the MCU 115 may initialize the multichannel ADC 112, and then the multichannel ADC 112 may transmit a high-speed connection request to the millimeter wave transmission module 113 after the clock synchronization is achieved based on the JESD204B protocol. One the other hand, the MCU 115 may also start timing in response to the clock-locking signal and generate a first connection instruction after a first preset period. For example, the first connection instruction may be a synchronization signal generated by the MCU 115 based on the JESD204B protocol, and be transmitted to a JESD204B receiving module of the multichannel ADC 112 after the first preset period. The connection request may be transmitted to the SOC 122 via the millimeter wave receiving module 121. Further, the SOC 122 may start timing in response to the connection request, and generate a second connection instruction corresponding to the first connection instruction based on the JESD204B protocol after a second preset period. For example, the second connection instruction may be a synchronization signal generated by the SOC 122 based on the JESD204B protocol, and be transmitted to a JESD204B receiving module of the SOC 122 after the second preset period.

Then, in response to the first connection instruction, the multichannel ADC 112 may generate link data and transmit the link data to the millimeter wave transmission module 113. The millimeter wave transmission module 113 may further transmit the link data to the SOC 122 via the millimeter wave receiving module 121. Further, the SOC 122 may confirm whether the first communication link is established based on the link data. If the SOC 122 confirms that the first communication link is established based on the link data, the establishment of the first communication link is completed. If the SOC 122 confirms that the first communication link is not established based on the link data, the laser emission module 123 may stop sending the clock signal, and the MCU 115 may initialize the signal converting device 11 after recognizing that phase-locked loop 116 is out of lock; after a period of time, the laser emission module 123 re-sends a new clock signal and transmit the new clock signal to the laser receiving module 114 to generate a new connection request; then, a new first connection instruction and a new second connection instruction may be generated based on the new connection request; further, new link data may be generated based on the new first connection instruction; the signal processing device 12 may re-confirm whether the first communication link is established based on the new link data until the establishment of the first communication link is completed.

In some embodiments, after the first communication link is established, the signal converting device 11 and the signal processing device 12 may maintain a wireless communication state. The SOC 122 may discard the invalid digital MR signals and send the remaining digital MR signals according to an instruction of the upper computer 30 to the upper computer 30. After the first communication link is established and before using the first communication link to transmit MR signals, the upper computer 30 may send an instruction to the SOC 122. After the instruction is received, the SOC 122 may parse and encapsulate the instruction, and issue a secondary instruction to the MCU 115. After receiving the secondary instruction, the MCU 115 may analyze the secondary instruction and control the one or more analog signal processors 111 to perform preprocessing operations (e.g., the amplifying operation, the filtering operation, the mixing frequency operation, etc.) on the analog MR signals collected by the receiver coil 20. In some embodiments, if the operating parameters of the analog signal filter and/or the analog signal gainer may be non-adjustable, the SOC 122 does not issue the secondary instruction to the MCU 115.

The JESD204B protocol may be described as an example for illustration. The preprocessed analog MR signals may be transmitted to the one or more ADCs 112, and the one or more ADC 112 may convert the analog MR signals into JESD204B protocol digital signals. The millimeter wave transmission module 113 may convert the JESD204B protocol digital signals into millimeter wave signals and transmit the millimeter wave signals to the signal processing device 12. The millimeter wave receiving module 121 of the signal processing device 12 may re-converts the millimeter wave signals into JESD204B protocol digital signals and transmits the JESD204B protocol digital signals to a high-speed digital interface of the SOC 122. After the SOC 122 parses the digital MR signals according to the JESD204B protocol, the SOC 122 may perform operations (e.g., a filter operation, a frequency conversion operation, a compression and package operation) on the digital MR signals, and then upload the processed digital MR signals to the upper computer 30. In some embodiments, the SOC 122 may process the digital MR signals using an AI algorithm such as a machine learning algorithm (e.g., a neural network algorithm, a deep learning algorithm). The upper computer 30 may perform data processing and generate an MR image.

According to the data transmission system 900, the signal converting device 11 and the signal processing device 12 may generate the first connection instruction and the second connection instruction corresponding to the first connection instruction, respectively. Further, in response to the first connection instruction, the signal converting device 11 may directly generate link data and transmit the link data to the signal processing device 12. That is, the signal processing device 12 does not generate and transmit a connection instruction to the signal converting device 11, thereby reducing the number of connections (e.g., only two connections) between the signal converting device 11 and the signal processing device 12. In addition, the clock signal in the data transmission system 900 is transmitted separately, which may have a relatively high clock quality (e.g., a relatively low phase jitter).

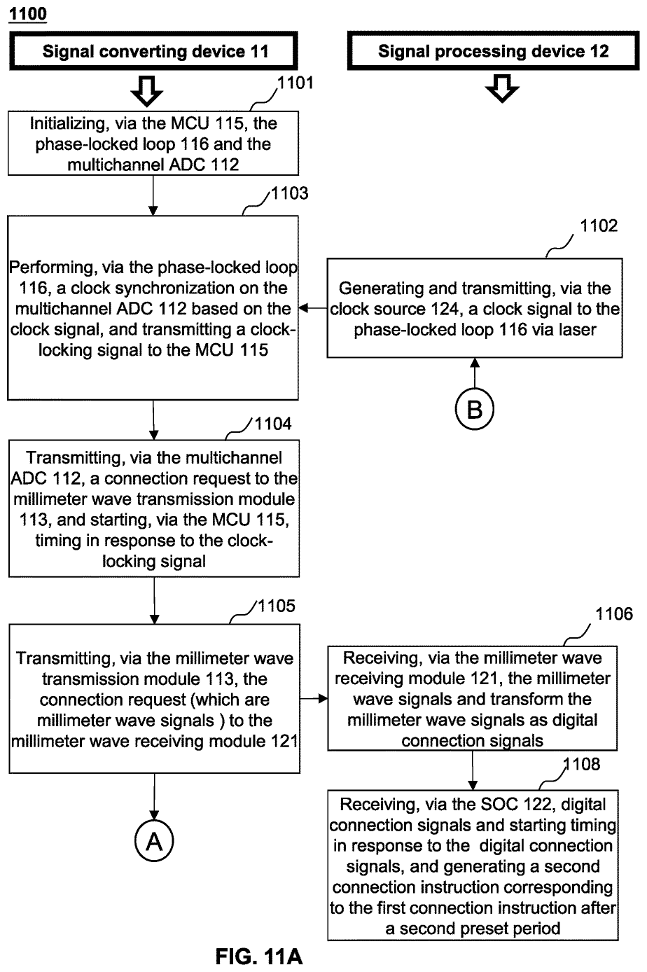

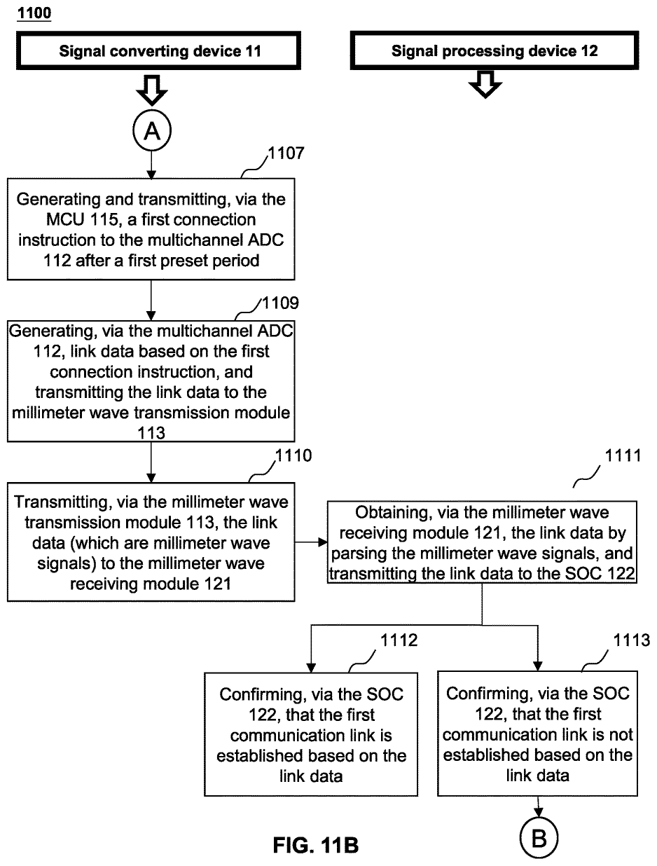

FIGS. 11A and 11B are flowcharts illustrating an exemplary process 1100 for establishing a first communication link according to some embodiments of the present disclosure. The process 1100 may be performed by the data transmission system 1000 in FIG. 10.

In 1101, the MCU 115 may initialize the phase-locked loop 116 and the multichannel ADC 112.

In 1102, the clock source 124 may generate and transmit a clock signal to the phase-locked loop 116 via laser.

In 1103, the phase-locked loop 116 may perform a clock synchronization on the multichannel ADC 112 based on the clock signal, and transmit a clock-locking signal to the MCU 115.

In 1104, the multichannel ADC 112 may transmit a connection request to the millimeter wave transmission module 113, and the MCU 115 may start timing in response to the clock-locking signal.

In 1105, the millimeter wave transmission module 113 may transmit the connection request (which are millimeter wave signals) to the millimeter wave receiving module 121.

In 1106, the millimeter wave receiving module 121 may receive the millimeter wave signals and transform the millimeter wave signals as digital connection signals.

In 1107, the MCU 115 may generate and transmit a first connection instruction to the multichannel ADC 112 after a first preset period.

In 1108, the SOC 122 may receive the digital connection signals and start timing in response to the digital connection signals, and generate a second connection instruction corresponding to the first connection instruction after a second preset period.

In 1109, the multichannel ADC 112 may generate link data based on the first connection instruction, and transmit the link data to the millimeter wave transmission module 113.

In 1110, the millimeter wave transmission module 113 may transmit the link data (which are millimeter wave signals) to the millimeter wave receiving module 121.

In 1111, the millimeter wave receiving module 121 may obtain the link data by parsing the millimeter wave signals, and transmit the link data to the SOC 122.

Then, the SOC 122 may perform operation 1112 or operation 1113. In 1112, the SOC 122 may confirm that the first communication link is established based on the link data. In 1113, the SOC 122 may confirm that the first communication link is not established based on the link data. If operation 1113 is performed, the clock signal transmitter 123 may stop sending the clock signal, and the MCU 115 may initialize the signal converting device 11 after recognizing that phase-locked loop 116 is out of lock; after a period of time, the clock signal transmitter 123 re-sends a new clock signal and transmit the new clock signal to the clock signal receiver 114 to generate a new connection request; then, a new first connection instruction and a new second connection instruction may be generated based on the new connection request; further, new link data may be generated based on the new first connection instruction; the signal processing device 12 may re-confirm whether the first communication link is established based on the new link data until the establishment of the first communication link is completed.

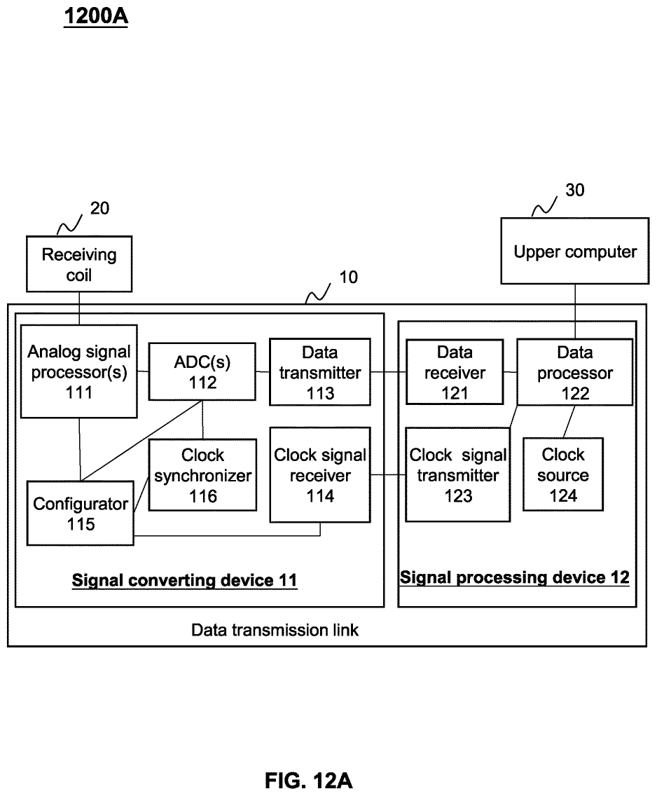

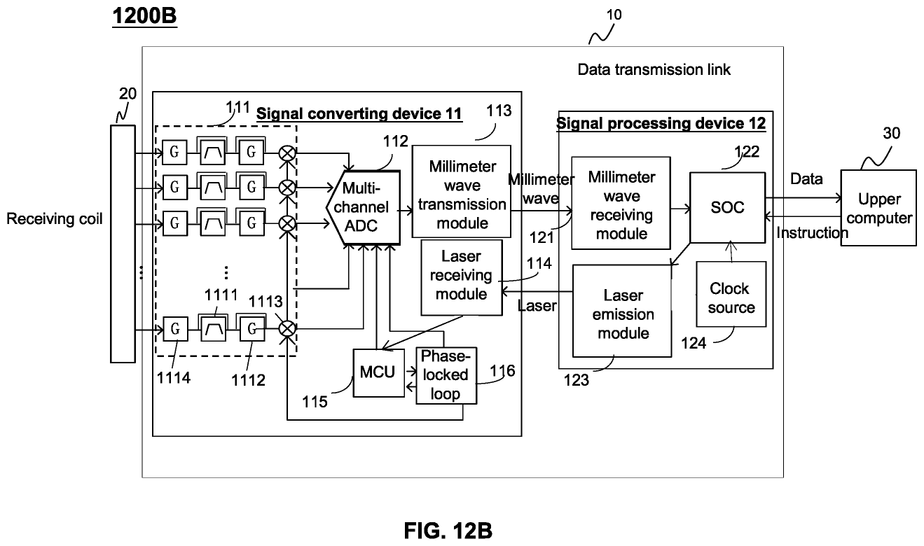

FIG. 12A is a schematic diagram illustrating an exemplary data transmission system 1200A according to some embodiments of the present disclosure. The data transmission system 1200A is an exemplary embodiment of the data transmission system 100 as described in connection with FIG. 1A.

As illustrated in FIG. 12A, the data transmission system 1200A may be similar to the data transmission system 900 as described in connection with FIG. 9, except that the clock source 124 is connected to the data processor 122 and is not connected to the clock signal transmitter 123, and the clock signal receive 114 is connected to the configurator 115 and is not connected to the clock synchronizer 116.

When establishing the first communication link in the data transmission system 1200A, the clock synchronization instruction may be transmitted in the form of high-speed data. Specifically, the clock source 124 may be configured to generate a clock signal, and the data processor 122 may be configured to generate the high-speed data based on the clock signal.

The second communication link may be established between the data processor 122 and the configurator 115. The configurator 115 may be configured to receive the high-speed data via the second communication link, restore the clock signal from the high-speed data, and transmit the clock signal to the clock synchronizer 116. The clock synchronizer 116 may perform the clock synchronization on the one or more ADCs 112 based on the clock signal, and transmit a clock-locking signal to the configurator 115. Further, the configurator 115 may be further configured to start timing in response to the clock-locking signal and generate a first connection instruction after the first preset period.

In some embodiments, the signal converting device 11 may further comprise one or more analog signal processors 111. Each analog signal processor 111 may be connected to the receiving coil 20, the configurator 115, and one of the one or more ADCs 112. The high-speed data further may further include one or more operating parameter values of the one or more analog signal processors 111. The configurator 115 may be further configured to restore the one or more operating parameter values from the high-speed data, and transmit the one or more operating parameter values to the one or more analog signal processors 111.

FIG. 12B is a schematic diagram illustrating an exemplary data transmission system 1200B according to some embodiments of the present disclosure. The data transmission system 1200B is an exemplary embodiment of the data transmission system 1200A. As illustrated in FIG. 12B, the one or more ADCs 112 includes one multichannel ADC, the data transmitter 113 is a millimeter wave transmission module, the clock signal receiver 114 is a laser receiving module, the configurator 115 is an MCU, the clock synchronizer 116 is a phase-locked loop, the data receiver 121 is a millimeter wave receiving module, the data processor 122 is a SOC, and the clock signal transmitter 123 is a laser emission module. The clock source 124 is similar to that in FIG. 10, the descriptions of which are not repeated. The operating parameters of the analog signal filter 1111 and the analog signal gainer 1112 of each analog signal processor 111 are adjustable.

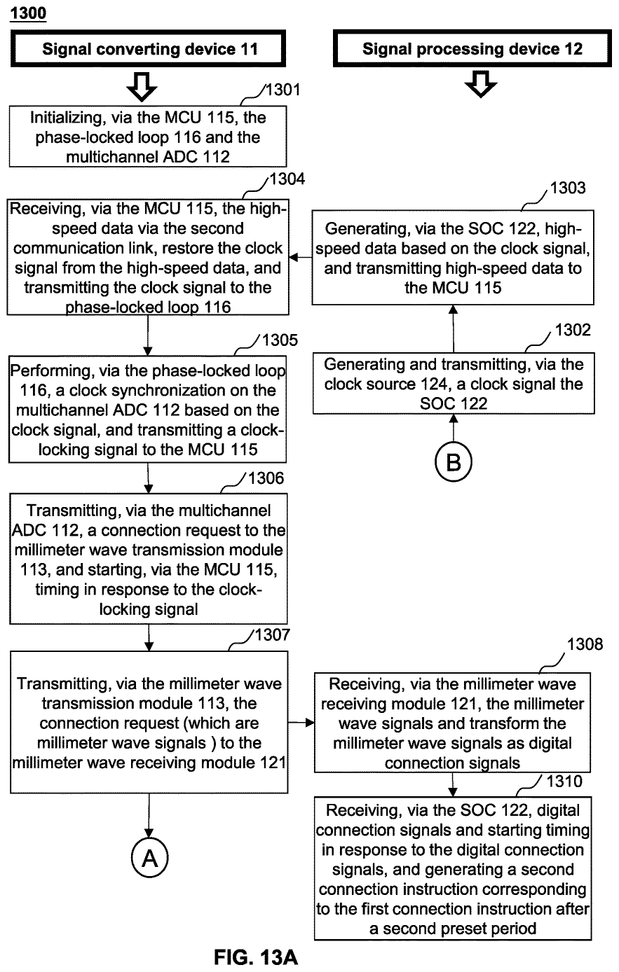

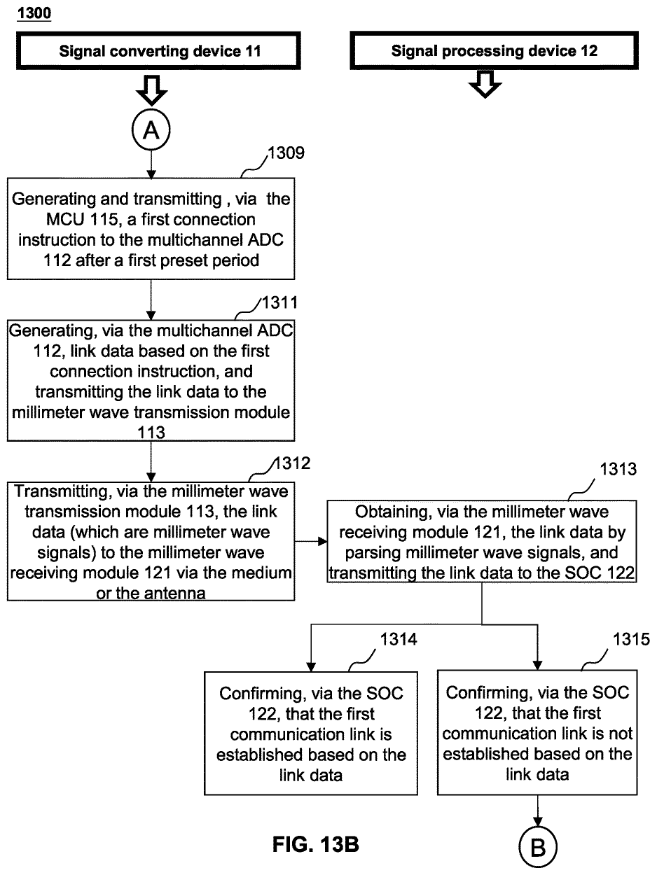

FIGS. 13A and 13B are flowcharts illustrating an exemplary process 1300 for establishing a first communication link according to some embodiments of the present disclosure. The process 1300 may be performed by the data transmission system 1200B in FIG. 12B.

In 1301, the MCU 115 may initialize the phase-locked loop 116 and the multichannel ADC 112.

In 1302, the clock source 124 may generate and transmit a clock signal to the SOC 122.

In 1303, the SOC 122 may generate high-speed data based on the clock signal, and transmit high-speed data to the MCU 115.

In 1304, the MCU 115 may receive the high-speed data via the second communication link, restore the clock signal from the high-speed data, and transmit the clock signal to the phase-locked loop 116.

In 1305, the phase-locked loop 116 may perform a clock synchronization on the multichannel ADC 112 based on the clock signal, and transmit a clock-locking signal to the MCU 115.

In 1306, the multichannel ADC 112 may transmit a connection request to the millimeter wave transmission module 113, and the MCU 115 may start timing in response to the clock-locking signal.

In 1307, the millimeter wave transmission module 113 may transmit the connection request (which are millimeter wave signals) to the millimeter wave receiving module 121.

In 1308, the millimeter wave receiving module 121 may receive the millimeter wave signals and transform the millimeter wave signals as digital connection signals.

In 1309, the MCU 115 may generate and transmit a first connection instruction to the multichannel ADC 112 after a first preset period.

In 1310, the SOC 122 may receive the digital connection signals and start timing in response to the digital connection signals, and generate a second connection instruction corresponding to the first connection instruction after a second preset period.

In 1311, the multichannel ADC 112 may generate link data based on the first connection instruction, and transmit the link data to the millimeter wave transmission module 113.

In 1312, the millimeter wave transmission module 113 may transmit the link data (which are millimeter wave signals) to the millimeter wave receiving module 121.

In 1313, the millimeter wave receiving module 121 may obtain the link data by parsing the millimeter wave signals, and transmit the link data to the SOC 122.

Then, the SOC 122 may perform operation 1314 or operation 1315. In 1314, the SOC 122 may confirm that the first communication link is established based on the link data. In 1315, the SOC 122 may confirm that the first communication link is not established based on the link data. If operation 1315 is performed, the SOC 122 may further perform 1302 to generate a new a clock signal and transmit the new a clock signal to the SOC 122.

As described elsewhere in the present disclosure, in the conventional data transmission systems, the signal converting device and the signal processing device need to be tightly integrated, often on the same circuit board, to achieve high data transmission rate, which limits the flexibility of arrangement of the signal converting device and the signal processing device, resulting in some problems such as increasing a volume, power consumption and a cost of an MR device, and making heat dissipation processing more difficult. Compared with the conventional data transmission systems, the signal converting device and the signal processing device in the data transmission systems and methods of the present disclosure do not need to be tightly integrated, which may improve the flexibility of arrangement of the signal converting device and the signal processing device while achieve high data transmission rate. In this way, the volume, the power consumption, and the cost of the MR device may be reduced, and the heat dissipation processing is easier, thereby having a high practicability. Moreover, according to some embodiments of the present disclosure, the clock synchronization instruction may be transmitted in the form of high-speed data, thereby improving efficiency of the data transmission. In some embodiments, the high-speed data may include both the clock signal and one or more operating parameter values for the one or more analog signal processors 111, which may save resources of the data transmission and further improve efficiency of the data transmission. In some embodiments, the connection instruction may be also transmitted in the form of high-speed data, which may further improve efficiency of the data transmission.

In addition, as we all know, if the clocks of the one or more ADCs 112 are not synchronous, the quality of MR imaging may be poor. According to some embodiments of the present discloser, the clocks of the one or more ADCs 112 may be synchronous by performing the clock synchronization on the one or more ADCs 112, which may improve imaging quality of the MR imaging.

It should be noted that the data transmission systems 100, 100B-100D, 300, 400, 600A, 600B, 900, 1000, 1200A, and 1200B, and the processes 200, 500, 700, 800,1100, and 1300 the descriptions thereof are provided for the purposes of illustration, and not intended to limit the scope of the present disclosure. For persons having ordinary skills in the art, various modifications and changes in the forms and details of the application of the above method and system may occur without departing from the principles of the present disclosure. However, those variations and modifications also fall within the scope of the present disclosure.

For example, a data transmission system disclosed herein (e.g., the data transmission system 100, 100B-100D, 300, 400, 600A, 600B, 900, 1000, 1200A, or 1200B) may include one or more additional components and/or one or more components of the data transmission system described above may be omitted. Additionally or alternatively, two or more components of the data transmission system may be integrated into a single component. A component of the data transmission system may be implemented on two or more sub-components. In some embodiments, the connections of components of the data transmission system may be available. As another example, the operations of the illustrated processes 200, 500, 700, 800,1100, and 1300 are intended to be illustrative. In some embodiments, the processes 200, 500, 700, 800,1100, and 1300 may be accomplished with one or more additional operations not described, and/or without one or more of the operations discussed. Additionally, the order in which the operations of the processes 200, 500, 700, 800,1100, and 1300, and regarding descriptions are not intended to be limiting.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” may mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, for example, an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±1%, ±5%, ±10%, or ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A data transmission system for magnetic resonance (MR) imaging, comprising:
a signal converting device configured to receive analog MR signals collected by a receiver coil in an MR scan, transform the analog MR signals into digital MR signals, generate transmission data based on the digital MR signals, and transmit the transmission data to a signal processing device via a first communication link between the signal converting device and the signal processing device; and
the signal processing device configured to receive the transmission data from the signal converting device via the first communication link and resolve the digital MR signals from the transmission data, wherein the signal converting device and the signal processing device are disposed at different circuit boards.

2. The data transmission system of claim 1, wherein to establish the first communication link,
the signal processing device is configured to transmit a clock synchronization instruction to the signal converting device via a second communication link between the signal converting device and the signal processing device,
in response to the clock synchronization instruction, the signal converting device is configured to perform clock synchronization on one or more analog-to-digital converters (ADCs) of the signal converting device and transmit a connection request to the signal processing device after the clock synchronization is achieved,
the signal processing device is configured to transmit a connection instruction to the signal converting device in response to the connection request, or the signal converting device is configured to generate the connection instruction,
in response to the connection instruction, the signal converting device is configured to generate link data and transmit the link data to the signal processing device,
the signal processing device is configured to confirm whether the first communication link is established based on the link data.

3. The data transmission system of claim 2, wherein the connection instruction is generated by the signal processing device and transmitted to the signal converting device.

4. The data transmission system of claim 3, wherein
the clock synchronization instruction is transmitted in the form of a clock signal,
the signal processing device includes a clock source configured to generate the clock signal,
the signal converting device includes a clock synchronizer,
the second communication link is established between the clock source and the clock synchronizer,
the clock synchronizer is configured to receive the clock signal via the second communication link and perform the clock synchronization on the one or more ADCs based on the clock signal.

5. The data transmission system of claim 4, wherein to perform the clock synchronization on the one or more ADCs, the clock synchronizer is further configured to:
for each of the one or more ADCs,
generate a second clock signal corresponding to the ADC based on the clock signal; and
transmit the second clock signal corresponding to the ADC to the ADC for performing the clock synchronization.

6. The data transmission system of claim 3, wherein the signal converting device includes:
a configurator connected to the signal processing device via a third communication link between the signal converting device and the signal processing device and configured to receive the connection instruction from the signal processing device via the third communication link;
the one or more ADCs connected to the configurator, wherein to generate the link data,
the configurator is configured to generate one or more synchronization signals in response to the connection instruction, and transmit the one or more synchronization signals to the one or more ADCs,
the one or more ADCs are configured to generate the link data in response to the one or more synchronization signals.

7. The data transmission system of claim 6, wherein
the signal converting device includes a data transmitter connected to the one or more ADCs, the signal processing device includes a data receiver, the first communication link is achieved by a wired or wireless connection between the data transmitter and the data receiver,
to generate the link data, the one or more ADCs are configured to obtain initial data, adjust the format and/or the phase of the initial data, and transform the adjusted initial data into the link data based on a digital protocol of the data receiver.

8. The data transmission system of claim 7, wherein the initial data are target digital MR signals converted from a portion of the analog MR signals.

9. The data transmission system of claim 8, wherein the signal converting device further comprises one or more analog signal processors each of which is connected to a receiving coil, the configurator, and one of the one or more ADCs,
the configurator is configured to receive a signal preprocessing instruction including one or more operating parameter values of the one or more analog signal processors from the signal processing device, and transmit the one or more operating parameter values to the one or more analog signal processors,
the one or more analog signal processors are configured to generate preprocessed analog MR signals by preprocessing the portion of the analog MR signals based on the one or more operating parameter values, and transmit the preprocessed analog MR signals to the one or more ADCs,
to convert the portion of the analog MR signals into the target digital MR signals, the one or more ADCs are configured to receive the preprocessed analog MR signals from the one or more analog signal processors and convert the preprocessed analog MR signals into the target digital MR signals.

10. The data transmission system of claim 3, wherein the clock synchronization instruction is transmitted in the form of high-speed data,
the signal processing device includes a clock source and a data processor connected to the clock source, the clock source being configured to generate a clock signal, and the data processor being configured to generate the high-speed data based on the clock signal,
the signal converting device includes a configurator,
the second communication link is established between the data processor and the configurator, and
the configurator is configured to receive the high-speed data via the second communication link, restore the clock signal from the high-speed data, and perform the clock synchronization on the one or more ADCs based on the clock signal.

11. The data transmission system of claim 10, wherein
the signal converting device further comprises one or more analog signal processors each of which is connected to a receiving coil, the configurator, and one of the one or more ADCs,
the high-speed data further includes one or more operating parameter values of the one or more analog signal processors,
the configurator is further configured to restore the one or more operating parameter values from the high-speed data, and transmit the one or more operating parameter values to the one or more analog signal processors.

12. The data transmission system of claim 2, wherein
the signal converting device is further configured to start timing in response to the clock synchronization instruction,
the connection instruction is generated by the signal converting device after a first preset period,
the signal processing device is further configured to start timing in response to the connection request, and generate a second connection instruction corresponding to the connection instruction after a second preset period.

13. The data transmission system of claim 12, wherein
the clock synchronization instruction is transmitted in the form of a clock signal,
the signal processing device includes a clock source configured to generate the clock signal,
the signal converting device includes a clock synchronizer and a configurator connected to the clock synchronizer,
the second communication link is established between the clock source and the clock synchronizer,
the clock synchronizer is configured to receive the clock signal from the clock source via the second communication link, perform the clock synchronization on the one or more ADCs based on the clock signal, and transmit a clock-locking signal to the configurator,
the configurator is configured to start timing in response to the clock-locking signal and generate the connection instruction after the first preset period.

14. The data transmission system of claim 13, wherein to generate the link data,
the configurator is configured to transmit the connection instruction to each of the one or more ADCs, and
the one or more ADCs are configured to generate the link data in response to the connection instruction.

15. The data transmission system of claim 13, wherein
the clock synchronizer is a phase-locked loop,
the one or more ADCs include one multichannel ADC,
the signal converting device further comprises one or more analog signal processors each of which includes a frequency mixer and is connected to a receiving coil, the phase-locked loop, and the multichannel ADC,
the phase-locked loop is configured to adjust a mixing frequency of the frequency mixer of each analog signal processor such that the frequency of preprocessed analog MR signals generated by each analog signal processor is within a preset frequency range corresponding to the multichannel ADC.

16. The data transmission system of claim 12, wherein the clock synchronization instruction is transmitted in the form of high-speed data,
the signal processing device includes a clock source and a data processor connected to the clock source, the clock source being configured to generate a clock signal, and the data processor being configured to generate the high-speed data based on the clock signal,
the signal converting device includes a clock synchronizer and a configurator connected to the clock synchronizer,
the second communication link is established between the configurator and the data processor,
the configurator is configured to receive the high-speed data via the second communication link, restore the clock signal from the high-speed data, and transmit the clock signal to the clock synchronizer,
the clock synchronizer is configured to perform the clock synchronization on the one or more ADCs based on the clock signal and transmit a clock-locking signal to the configurator,
the configurator is further configured to start timing in response to the clock-locking signal and generate the connection instruction after the first preset period.

17. The data transmission system of claim 16, wherein
the signal converting device further comprises one or more analog signal processors each of which is connected to a receiving coil, the configurator, and one of the one or more ADCs,
the high-speed data further includes one or more operating parameter values of the one or more analog signal processors,
the configurator is further configured to restore the one or more operating parameter values from the high-speed data, and transmit the one or more operating parameter values to the one or more analog signal processors.

18. The data transmission system of claim 1, wherein before the first communication link is established,
the signal processing device is configured to transmit a clock synchronization instruction to the signal converting device via a second communication link between signal converting device and the signal processing device,
in response to the clock synchronization instruction, the signal converting device is configured to start timing, perform clock synchronization on one or more ADCs, and transmit a connection request to the signal processing device after the clock synchronization is achieved,
after a first preset period, the signal converting device is further configured to generate a connection instruction, generate link data in response to the connection instruction, and transmit the link data to the signal processing device,
the signal processing device is configured to start timing in response to the connection request, generate a second connection instruction corresponding to the connection instruction after a second preset period, and confirm whether the first communication link is established based on the link data.

19. (canceled)

20. The data transmission system of claim 1, wherein the signal processing device is disposed on or within a gantry of an MR device.

21. (canceled)

22. A method for establishing a first communication link in magnetic resonance (MR) imaging, comprising:
by a signal processing device, transmitting a clock synchronization instruction to a signal converting device via a second communication link between the signal converting device and the signal processing device;
by the signal converting device, performing clock synchronization on one or more analog-to-digital converters (ADCs) of the signal converting device and transmitting a connection request to the signal processing device after the clock synchronization is achieved;
transmitting a connection instruction from the signal processing device to the signal converting device in response to the connection request, or generating the connection instruction by the signal converting device;
by the signal converting device, generating link data in response to the connection instruction and transmitting the link data to the signal processing device;
by the signal processing device, confirming whether the first communication link is established based on the link data.

23-26. (canceled)
